(19) United States
(12) Patent Application Publication
(10) Pub. No.: US 2024/0317866 A1
(43) Pub. Date: Sep. 26, 2024

(54) Use of Anti-EGFR/Anti-Met Antibody to Treat Gastric or Esophageal Cancer

(71) Applicant: Janssen Biotech, Inc., Horsham, PA (US)

(72) Inventors: Joshua Curtin, Jenkintown, PA (US); Roland Knoblauch, Philadelphia, PA (US); Meena Thayu, Bryn Mawr, PA (US)

(21) Appl. No.: 18/216,026

(22) Filed: Jun. 29, 2023

Related U.S. Application Data
(60) Provisional application No. 63/357,218, filed on Jun. 30, 2022.

(51) Int. Cl. C07K 16/28; A61K 9/00; A61K 39/00; A61K 45/06; A61P 35/00

(52) U.S. Cl. CPC C07K 16/2863; A61K 9/0019; A61K 45/06; A61P 35/00; A61K 2039/505; A61K 2039/54; A61K 2039/545; C07K 2317/31; C07K 2317/565

(57) ABSTRACT

The present disclosure provides methods of treating gastric or esophageal cancer in a subject in need thereof by administering a therapeutically effective amount of a bispecific anti-epidermal growth factor receptor (EGFR)/hepatocyte growth factor receptor (c-Met) antibody.

### CROSS-REFERENCE TO RELATED APPLICATIONS

This application claims the benefit of U.S. Provisional Patent Application No. 63/357,218, filed Jun. 30, 2022, the disclosure of which is herein incorporated by reference in its entirety.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6733USNP1SEQLIST.xml”, creation date of Jun. 28, 2023, and a size of 20 kilobytes (KB). This sequence listing submitted is part of the specification and is herein incorporated by reference in its entirety.

### FIELD

The present disclosure relates to methods of treating gastric or esophageal cancer with a bispecific anti-epidermal growth factor receptor (EGFR)/hepatocyte growth factor receptor (c-Met) antibody.

### BACKGROUND

Cancer is a leading cause of death worldwide. Gastric cancer (GC) is the fifth most common cancer worldwide and over 1 million new cases were reported worldwide in 2018. Gastric cancer is highly prevalent in Asian countries accounting for approximately 75% of new cases in 2018 and is the third (male)/fourth (female) most prevalent cancer in Japan. Majority of patients exhibit adenocarcinoma histology. The treatment regimen depends on the type of cancer (e.g., histology), the stage of the cancer at diagnosis, and the presence of molecular biomarkers (e.g., human epidermal growth factor receptor 2 (HER2) amplification, programmed death-ligand 1 (PD-L1) expression, and microsatellite instability). Treatment commonly includes surgery and chemotherapy. In patients with metastatic disease, National Comprehensive Cancer Network (NCCN) guidelines recommend HER2, programmed death-ligand 1 (PD-L1), and microsatellite instability testing (NCCN Guidelines 2020). The first line treatment generally includes fluoropyrimidine plus cisplatin or oxaliplatin plus trastuzumab (depending on HER2 status). The observed overall response rate for the guideline-recommended initial treatment varies widely (e.g., between 35% to 68% for the combination therapy with oral fluoropyrimidine (S-1) in Japan (Bang 2010; Kurokawa 2014)). Available treatment options after first line treatment are limited. The NCCN recommendations are chemotherapy as a single agent, ramucirumab plus paclitaxel, immune checkpoint inhibitor, or fluorouracil plus irinotecan. However, the overall response rate for the second and third line treatments is limited and the median progression-free survival is very low.

Esophageal cancer (EC) is the eighth most common cancer worldwide and ranked sixth among all cancers in mortality in 2018. Similar to GC, EC is highly prevalent in Asian countries accounting for over 75% of new cases in 2018. Squamous cell carcinoma and adenocarcinoma are two major histologies of primary ECs. However, predominant EC histology varies between geographical regions. The predominant histology observed among the Caucasian population is adenocarcinoma whereas squamous histology predominates Asian countries. Treatment commonly includes surgery, radiation therapy, chemoradiation therapy, and chemotherapy. The NCCN guideline recommendations for the first line therapies are identical to GC regimens due to the nature of histological similarities (NCCN Guidelines 2020). Clinical data of esophageal squamous cell carcinoma, which is the predominant histology in Asian countries, are limited to mostly Phase 2 studies. The reported overall response rate of chemotherapy as a single agent is 15%˜40%. Other recommended treatments include fluorouracil plus cisplatin and PD-(L)1 inhibitor, pembrolizumab. Yet, the observed overall response rates to these therapies are quite limited.

As such, there is an unmet need in the art for improved therapies for the treatment of gastric or esophageal cancer.

### SUMMARY

In various aspects, provided herein are methods of treating gastric or esophageal cancer in a subject in need thereof, comprising administering to the subject a therapeutically effective amount of a bispecific anti-epidermal growth factor receptor (EGFR)/hepatocyte growth factor receptor (c-Met) antibody.

In one aspect, the present disclosure provides a method of treating gastric cancer in a subject in need thereof, comprising administering to the subject a therapeutically effective amount of a bispecific anti-EGFR/c-Met antibody.

In some embodiments of the method for treating gastric cancer, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments of the method for treating gastric cancer, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

In some embodiments of the method for treating gastric cancer, the bispecific anti-EGFR/c-Met antibody is an IgG1 isotype.

In some embodiments of the method for treating gastric cancer, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments of the method for treating gastric cancer, the bispecific anti-EGFR/c-Met antibody comprises a biantennary glycan structure with a fucose content of about between 1% to about 15%.

In some embodiments of the method for treating gastric cancer, the bispecific anti-EGFR/c-Met antibody is administered intravenously or subcutaneously to the subject.

In some embodiments of the method for treating gastric cancer, the bispecific anti-EGFR/c-Met antibody is administered at a dose of between about 350 mg to about 3400 mg.

In some embodiments of the method for treating gastric cancer, the bispecific anti-EGFR/c-Met antibody is administered at a dose of about 350 mg. 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg. 1800 mg. 1850 mg. 1900 mg, 1950 mg, 2000 mg, 2100 mg, 2200 mg, 2240 mg, 2300 mg, 2400 mg, 2500 mg, 2600 mg, 2700 mg, 2800 mg, 2900 mg, 3000 mg, 3100 mg, 3200 mg, 3300 mg. 3360 mg, or 3400 mg.

In some embodiments of the method for treating gastric cancer, the bispecific anti-EGFR/c-Met antibody is administered at a dose of 1050 mg. In some embodiments, the bispecific anti-EGFR/c-Met antibody is administered at a dose of 1400 mg. In some embodiments, the bispecific anti-EGFR/c-Met antibody is administered at a dose of 1600 mg. In some embodiments, the bispecific anti-EGFR/c-Met antibody is administered at a dose of 1750 mg. In some embodiments, the bispecific anti-EGFR/c-Met antibody is administered at a dose of 2100 mg. In some embodiments, the bispecific anti-EGFR/c-Met antibody is administered at a dose of 2240 mg. In some embodiments, the bispecific anti-EGFR/c-Met antibody is administered at a dose of 2400 mg. In some embodiments, the bispecific anti-EGFR/c-Met antibody is administered at a dose of 3360 mg.

In some embodiments of the method for treating gastric cancer, the bispecific anti-EGFR/c-Met antibody is administered subcutaneously or intradermally to the subject. In some embodiments, the bispecific anti-EGFR/c-Met antibody is administered subcutaneously or intradermally at a dose sufficient to achieve a therapeutic effect in the subject.

In some embodiments of the method for treating gastric cancer, the bispecific anti-EGFR/c-Met antibody is administered intravenously to the subject. In some embodiments, the bispecific anti-EGFR/c-Met antibody is administered intravenously at a dose sufficient to achieve a therapeutic effect in the subject.

In some embodiments of the method for treating gastric cancer, the bispecific anti-EGFR/c-Met antibody is administered twice a week, once a week, once in two weeks, once in three weeks or once in four weeks. In some embodiments, the bispecific anti-EGFR/c-Met antibody is administered once a week for four weeks and once in two weeks thereafter. In some embodiments, the first dose of the bispecific anti-EGFR/c-Met antibody is administered over two days.

In some embodiments of the method for treating gastric cancer, one or more cells of the gastric cancer express EGFR and/or cMet.

In some embodiments of the method for treating gastric cancer, the subject has received a prior treatment. In some embodiments, the prior treatment comprises a chemotherapy, a targeted therapy, an immunotherapy, surgery, radiation therapy, chemoradiation therapy, or a combination thereof. In some embodiments, the chemotherapy comprises a fluoropyrimidine-based chemotherapy, a platinum-based chemotherapy, paclitaxel, irinotecan, or a combination thereof. In some embodiments, the fluoropyrimidine is 5-fluorouracil or capecitabine. In some embodiments, the platinum-based chemotherapy is cisplatin, oxaliplatin, carboplatin, or nedaplatin. In some embodiments, the targeted therapy comprises an anti-HER2 therapy or anti-VEGF/VEGFR therapy. In some embodiments, the anti-HER2 therapy comprises trastuzumab. In some embodiments, the anti-VEGF/VEGFR therapy comprises bevacizumab or ramucirumab.

In some embodiments of the method for treating gastric cancer, the method further comprises administering at least one additional therapeutic to the subject. In some embodiments, the additional therapeutic comprises a glucocorticosteroid, antihistamine, antipyretic, H2-antagonist, antiemetic, opiate, or any combination thereof.

In some embodiments of the method for treating gastric cancer, the gastric cancer is an advanced or metastatic cancer.

In some embodiments of the method for treating gastric cancer, the subject is human.

In one aspect, the present disclosure provides a method of treating esophageal cancer in a subject in need thereof, comprising administering to the subject a therapeutically effective amount of a bispecific anti-EGFR/c-Met antibody.

In some embodiments of the method for treating esophageal cancer, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments of the method for treating esophageal cancer, the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

In some embodiments of the method for treating esophageal cancer, the bispecific anti-EGFR/c-Met antibody is an IgG1 isotype.

In some embodiments of the method for treating esophageal cancer, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In some embodiments of the method for treating esophageal cancer, the bispecific anti-EGFR/c-Met antibody comprises a biantennary glycan structure with a fucose content of about between 1% to about 15%.

In some embodiments of the method for treating esophageal cancer, the bispecific anti-EGFR/c-Met antibody is administered intravenously or subcutaneously to the subject.

In some embodiments of the method for treating esophageal cancer, the bispecific anti-EGFR/c-Met antibody is administered at a dose of between about 350 mg to about 3400 mg.

In some embodiments of the method for treating esophageal cancer, the bispecific anti-EGFR/c-Met antibody is administered at a dose of about 350 mg, 700 mg, about 750 mg, about 800 mg, about 850 mg, 900 mg. 950 mg, 1000 mg, 1050 mg, 1100 mg, 1150 mg, 1200 mg, 1250 mg. 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2100 mg, 2200 mg, 2240 mg, 2300 mg, 2400 mg, 2500 mg, 2600 mg, 2700 mg, 2800 mg, 2900 mg, 3000 mg, 3100 mg, 3200 mg, 3300 mg, 3360 mg, or 3400 mg.

In some embodiments of the method for treating esophageal cancer, the bispecific anti-EGFR/c-Met antibody is administered at a dose of 1050 mg. In some embodiments, the bispecific anti-EGFR/c-Met antibody is administered at a dose of 1400 mg. In some embodiments, the bispecific anti-EGFR/c-Met antibody is administered at a dose of 1600 mg. In some embodiments, the bispecific anti-EGFR/c-Met antibody is administered at a dose of 1750 mg. In some embodiments, the bispecific anti-EGFR/c-Met antibody is administered at a dose of 2100 mg. In some embodiments, the bispecific anti-EGFR/c-Met antibody is administered at a dose of 2240 mg. In some embodiments, the bispecific anti-EGFR/c-Met antibody is administered at a dose of 2400 mg. In some embodiments, the bispecific anti-EGFR/c-Met antibody is administered at a dose of 3360 mg.

In some embodiments of the method for treating esophageal cancer, the bispecific anti-EGFR/c-Met antibody is administered subcutaneously or intradermally to the subject. In some embodiments, the bispecific anti-EGFR/c-Met antibody is administered subcutaneously or intradermally at a dose sufficient to achieve a therapeutic effect in the subject.

In some embodiments of the method for treating esophageal cancer, the bispecific anti-EGFR/c-Met antibody is administered intravenously to the subject. In some embodiments, the bispecific anti-EGFR/c-Met antibody is administered intravenously at a dose sufficient to achieve a therapeutic effect in the subject.

In some embodiments of the method for treating esophageal cancer, the bispecific anti-EGFR/c-Met antibody is administered twice a week, once a week, once in two weeks, once in three weeks or once in four weeks. In some embodiments, the bispecific anti-EGFR/c-Met antibody is administered once a week for four weeks and once in two weeks thereafter. In some embodiments, the first dose of the bispecific anti-EGFR/c-Met antibody is administered over two days.

In some embodiments of the method for treating esophageal cancer, one or more cells of the esophageal cancer express EGFR and/or cMet.

In some embodiments of the method for treating esophageal cancer, the subject has received a prior treatment. In some embodiments, the prior treatment comprises a chemotherapy, a targeted therapy, an immunotherapy, surgery, radiation therapy, chemoradiation therapy, or a combination thereof. In some embodiments, the chemotherapy comprises a fluoropyrimidine-based chemotherapy, a platinum-based chemotherapy, paclitaxel, irinotecan, or a combination thereof. In some embodiments, the fluoropyrimidine is 5-fluorouracil or capecitabine. In some embodiments, the platinum-based chemotherapy is cisplatin, oxaliplatin, carboplatin, or nedaplatin. In some embodiments, the targeted therapy comprises an anti-HER2 therapy or anti-VEGF/VEGFR therapy. In some embodiments, the anti-HER2 therapy comprises trastuzumab. In some embodiments, the anti-VEGF/VEGFR therapy comprises bevacizumab or ramucirumab.

In some embodiments of the method for treating esophageal cancer, the subject is treatment naïve.

In some embodiments of the method for treating esophageal cancer, the method further comprises administering at least one additional therapeutic to the subject. In some embodiments, the additional therapeutic is a glucocorticosteroid, antihistamine, antipyretic, H2-antagonist, antiemetic, opiate, or any combination thereof.

In some embodiments of the method for treating esophageal cancer, the esophageal cancer is an advanced or metastatic cancer.

In some embodiments of the method for treating esophageal cancer, the subject is human.

### BRIEF DESCRIPTION OF THE DRAWINGS

FIG. 1 depicts one aspect of the Phase 2 study described in Example 1.

FIGS. 2A-2L show tumor growth curves (expressed as Mean±SEM) over time in a series of esophageal patient-derived xenografts (PDX) models.

FIGS. 3A-3F show tumor growth curves (expressed as Mean±SEM) over time in a series of gastric PDX models.

FIG. 4 shows relative EGFR and c-Met H-scores together with anti-tumor activity of amivantamab in a series of esophageal PDX models.

FIG. 5 shows relative EGFR and c-Met H-scores together with anti-tumor activity of amivantamab in a series of gastric PDX models.

FIG. 6 shows the overall response for gastric cancer patients in response evaluable population.

FIG. 7 shows the overall response for gastric cancer patients in all treated population.

FIG. 8 shows the overall response for esophageal cancer patients in response evaluable population.

FIG. 9 shows the overall response for esophageal cancer patients in all treated population.

FIG. 10 shows the overall response for esophageal cancer in patients treated with 1750 mg amivantamab in response evaluable population.

### DETAILED DESCRIPTION

Receptor tyrosine kinases (RTK) are involved in the regulation of many processes in mammalian development, cell function, and tissue homeostasis. Dysregulation of RTKs has been implicated in the development of numerous human cancers, and various RTKs are targets for both approved and experimental anticancer therapies.

Epidermal growth factor receptor (EGFR), an RTK in the HER family, is normally expressed in tissues of epithelial, mesenchymal, and neuronal origin. Binding of any of its 7 ligands, including EGF, induces diverse cellular responses, including differentiation, proliferation, migration, and survival (Olayioye 2000). The mesenchymal-epithelial transition factor (cMet or MET) receptor is also an RTK, expressed in normal epithelial cells (Prat 1991), with a role in growth and homeostasis, including embryonic development, angiogenesis, and wound healing (Sattler 2011). cMet is activated by a single specific ligand, hepatocyte growth factor, also known as scatter factor.

Overexpression and mutations of the EGFR and cMet receptors have been linked to tumorigenesis and malignancy, as well as poor prognosis in several types of cancer (Birchmeier 2003; Hyner 2005; Yano 2003). It is reported that approximately 25% and 50% of gastric cancer (GC) patients express EGFR or cMet, respectively (Fuse 2016). Likewise, approximately 60% to 70% and 45% to 70% of esophageal cancer (EC) patients express EGFR or cMet, respectively (Hanawa 2006; Gibault 2005). The expression of EGFR or cMet has been implicated as a poor prognostic factor in GC (Aydin 2014; Gao 2013; Galizia 2007; Atmaca 2012; Fuse 2016) and EC (Wang 2007; Brand 2011; Ozawa 2015).

Despite numerous agents targeting EGFR (including anti-EGFR antibodies and EGFR tyrosine kinase inhibitors (TKIs)) having been used as a standard of care for many cancers, including colorectal cancer, non-small cell lung cancer (NSCLC), and head and neck cancer, no EGFR-directed therapy is available for gastric or ECs. Previous studies have failed to show efficacy of cetuximab and panitumumab, anti-EGFR antibodies, for the treatment of GC or gefitinib, EGFR tyrosine kinase inhibitor, in EC in non-biomarker selected population (Lordick 2013; Waddell 2013; Dutton 2014). A more recent study evaluating nimotuzumab, anti-EGFR antibody, in combination with irinotecan for GC patients with EGFR expression has also been shown to be unsuccessful (Satoh 2015). The clinical experience of cMet-targeted therapy is less extensive. The studies evaluating the efficacy of rilotumumab or onartuzumab, anti-cMet antibodies, in GC failed to show clinical benefit.

The present disclosure provides methods and compositions useful for treating gastric or esophageal cancer by targeting both EGFR and cMet.

### Definitions

It is to be understood that the terminology used herein is for describing particular embodiments only and is not intended to be limiting. Unless defined otherwise, all technical and scientific terms used herein have the same meaning as commonly understood by one of ordinary skill in the art to which the invention pertains.

Although any methods and materials similar or equivalent to those described herein may be used in the practice for testing of the present invention, exemplary materials and methods are described herein. In describing and claiming the present invention, the following terminology will be used.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“Co-administration,” “administration with,” “administration in combination with,” “in combination with” or the like, encompass administration of the selected therapeutics or drugs to a single patient, and are intended to include treatment regimens in which the therapeutics or drugs are administered by the same or different route of administration or at the same or different time.

“Treat”, “treating” or “treatment” of a disease or disorder such as cancer refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Prevent”, “preventing”, “prevention”, or “prophylaxis” of a disease or disorder means preventing that a disorder occurs in subject.

“Responsive”, “responsiveness” or “likely to respond” refers to any kind of improvement or positive response, such as alleviation or amelioration of one or more symptoms, diminishment of extent of disease, stabilized (i.e., not worsening) state of disease, preventing spread of disease, delay or slowing of disease progression, amelioration or palliation of the disease state, and remission (whether partial or total), whether detectable or undetectable.

“Therapeutically effective amount” refers to an amount effective, at doses and for periods of time necessary, to achieve a desired therapeutic result. A therapeutically effective amount may vary depending on factors such as the disease state, age, sex, and weight of the individual, and the ability of a therapeutic or a combination of therapeutics to elicit a desired response in the individual. Exemplary indicators of an effective therapeutic or combination of therapeutics that include, for example, improved well-being of the patient, decrease or shrinkage of the size of a tumor, arrested or slowed growth of a tumor, and/or absence of metastasis of cancer cells to other locations in the body.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” are used interchangeably herein.

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Cancer” refers to an abnormal growth of cells which tend to proliferate in an uncontrolled way and, in some cases, to metastasize (spread) to other areas of a patient's body.

“EGFR or c-Met or MET expressing cancer” refers to cancer that has detectable expression of EGFR or c-Met or has EGFR or c-Met mutation or amplification. EGFR or c-Met expression, amplification and mutation status can be detected using know methods, such as sequencing, fluorescent in situ hybridization, immunohistochemistry, flow cytometry or western blotting.

“Epidermal growth factor receptor” or “EGFR” refers to the human EGFR (also known as HER1 or ErbB1 (Ullrich et al., Nature 309:418-425, 1984)) having the amino acid sequence shown in GenBank accession number NP_005219, as well as naturally occurring variants thereof.

“Hepatocyte growth factor receptor” or “c-Met” or “MET” as used herein refers to the human c-Met having the amino acid sequence shown in GenBank Accession No: NP_001120972 and natural variants thereof.

“Bispecific anti-EGFR/c-Met antibody” or “bispecific EGFR/c-Met antibody” refers to a bispecific antibody having a first domain that specifically binds EGFR and a second domain that specifically binds c-Met. The domains specifically binding EGFR and c-Met are typically VH/VL pairs, and the bispecific anti-EGFR/c-Met antibody is monovalent in terms of binding to EGFR and c-Met.

“Specific binding” or “specifically binds” or “specifically binding” or “binds” refer to an antibody binding to an antigen or an epitope within the antigen with greater affinity than for other antigens. Typically, the antibody binds to the antigen or the epitope within the antigen with an equilibrium dissociation constant (K_{D}) of about 5×10⁻⁸ M or less, for example about 1×10⁻⁹ M or less, about 1×10⁻¹⁰ M or less, about 1×10⁻¹¹ M or less, or about 1×10⁻¹² M or less, typically with the Kp that is at least one hundred-fold less than its Kp for binding to a non-specific antigen (e.g., BSA, casein). The dissociation constant may be measured using known protocols. Antibodies that bind to the antigen or the epitope within the antigen may, however, have cross-reactivity to other related antigens, for example to the same antigen from other species (homologs), such as human or monkey, for example Macaca fascicularis (cynomolgus, cyno) or Pan troglodytes (chimpanzee, chimp). While a monospecific antibody binds one antigen or one epitope, a bispecific antibody binds two distinct antigens or two distinct epitopes.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Complementarity determining regions” (CDR) are antibody regions that bind an antigen. CDRs may be defined using various delincations such as Kabat (Wu et al. (1970) J Exp Med 132: 211-50) (Kabat et al., Sequences of Proteins of Immunological Interest, 5th Ed. Public Health Service, National Institutes of Health, Bethesda, Md., 1991), Chothia (Chothia et al. (1987) J Mol Biol 196: 901-17), IMGT (Lefranc et al. (2003) Dev Comp Immunol 27: 55-77) and AbM (Martin and Thornton (1996) J Bmol Biol 263: 800-15). The correspondence between the various delincations and variable region numbering are described (see e.g., Lefranc et al. (2003) Dev Comp Immunol 27: 55-77; Honegger and Pluckthun, (2001) J Mol Biol 309:657-70; International ImMunoGeneTics (IMGT) database; Web resources, http://imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (2), based on the amino acid sequences of their constant domains.

“Antigen binding fragment” refers to a portion of an immunoglobulin molecule that binds an antigen. Antigen binding fragments may be synthetic, enzymatically obtainable or genetically engineered polypeptides and include the VH, the VL, the VH and the VL, Fab, F(ab′)2, Fd and Fv fragments, domain antibodies (dAb) consisting of one VH domain or one VL domain, shark variable IgNAR domains, camelized VH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3. VH and VL domains may be linked together via a synthetic linker to form various types of single chain antibody designs where the VH/VL domains may pair intramolecularly, or intermolecularly in those cases when the VH and VL domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804 and WO1992/01047.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Recombinant” refers to DNA, antibodies and other proteins that are prepared, expressed, created or isolated by recombinant means when segments from different sources are joined to produce recombinant DNA, antibodies or proteins.

“Bispecific” refers to an antibody that specifically binds two distinct antigens or two distinct epitopes within the same antigen. The bispecific antibody may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Antagonist” or “inhibitor” refers to a molecule that, when bound to a cellular protein, suppresses at least one reaction or activity that is induced by a natural ligand of the protein. A molecule is an antagonist when the at least one reaction or activity is suppressed by at least about 20%, 30%, 40%, 45%, 50%, 55%, 60%, 65%, 70%, 75%, 80%, 85%, 90%, 95%, or 100% more than the at least one reaction or activity suppressed in the absence of the antagonist (e.g., negative control), or when the suppression is statistically significant when compared to the suppression in the absence of the antagonist.

“PD-(L)1 axis inhibitor” refers to a molecule that inhibits PD-1 downstream signaling. PD-(L)1 axis inhibitor may be a molecule that binds PD-1, PD-L1 or PD-L2.

“Low fucose” or “low fucose content” as used in the application refers to antibodies with fucose content of about between 1%-15%.

“Normal fucose” or “normal fucose content” as used herein refers to antibodies with fucose content of about over 50%, typically about over 80% or over 85%.

### Methods of the Disclosure

One aspect of the disclosure provides a method of treating gastric or esophageal cancer in a subject in need thereof, comprising administering to the subject a therapeutically effective amount of a bispecific anti-EGFR/c-Met antibody.

In some embodiments, the disclosure provides a method of treating gastric cancer in a subject in need thereof, comprising administering to the subject a therapeutically effective amount of a bispecific anti-EGFR/c-Met antibody.

Gastric cancer referred to herein also includes esophagogastric junction (GEJ) cancer. In some embodiments, the gastric cancer is adenocarcinoma. In some embodiments, the gastric cancer is an advanced or metastatic cancer. For example, the gastric cancer may have metastasized to the esophagus, the small intestine, lymph nodes, organs, bones, or combinations thereof.

In some embodiments, the disclosure provides a method of treating esophageal cancer in a subject in need thereof, comprising administering to the subject a therapeutically effective amount of a bispecific anti-EGFR/c-Met antibody.

In some embodiments, the esophageal cancer is adenocarcinoma. In some embodiments, the esophageal cancer is squamous cell carcinoma. In some embodiments, the esophageal cancer is an advanced or metastatic cancer. For example, the esophageal cancer may have metastasized to the lung, the small intestine, lymph nodes, organs, bones, or combinations thereof.

In some embodiments, the subject has received prior treatment. The prior treatment may include a chemotherapy, a targeted therapy, an immunotherapy, surgery, radiation therapy, chemoradiation therapy, or a combination thereof.

In some embodiments, the chemotherapy is a fluoropyrimidine-based chemotherapy, such as 5-fluorouracil or capecitabine.

In some embodiments, the chemotherapy is a platinum-based chemotherapy. Exemplary platinum-based chemotherapies include, but not limited to, cisplatin, oxaliplatin, carboplatin, or nedaplatin.

Additional examples of chemotherapy may include taxanes (e.g., paclitaxel, docetaxel), topoisomerase inhibitors (e.g., irinotecan, camptothecin), or a combination thereof.

In some embodiments, the targeted therapy is an anti-HER2 therapy or anti-VEGF/VEGFR therapy. Non-limiting examples of anti-HER2 therapy include trastuzumab. Non-limiting examples of anti-VEGF/VEGFR therapy include bevacizumab or ramucirumab.

In some embodiments, the prior treatment comprises an immunotherapy such as checkpoint inhibitors. In some embodiments, the immunotherapy comprises a PD-(L)1 axis inhibitor, or a CTLA-4 inhibitor. Non-limiting examples of PD-(L)1 axis inhibitors include atezolizumab, nivolumab, pembrolizumab, camrelizumab, and tislelizumab. Non-limiting examples of CTLA-4 inhibitors include ipilimumab.

In some embodiments, the prior treatment comprises an anti-VEGF/VEGFR therapy. Non-limiting examples of anti-VEGF/VEGFR therapy include bevacizumab and ramucirumab.

In some embodiments, the prior treatment comprises fluoropyrimidine and cisplatin.

In some embodiments, the prior treatment comprises oxaliplatin and trastuzumab.

In some embodiments, the prior treatment comprises ramucirumab and paclitaxel.

In some embodiments, the prior treatment comprises fluorouracil and irinotecan.

In some embodiments, the prior treatment comprises fluorouracil and cisplatin.

In some embodiments, the subject is treatment naïve.

In some embodiments, one or more cells of the gastric or esophageal cancer express EGFR and/or cMet. EGFR or c-Met expression can be detected using know methods, such as fluorescent in situ hybridization, immunohistochemistry (IHC), flow cytometry or western blotting.

In some embodiments, expression of EGFR and/or cMet is detected using immunohistochemistry (IHC), which measures EGFR and/or cMet protein levels on the cell surface. As a non-limiting example, a membrane staining intensity score (0, 1+, 2+, or 3+) may be determined for each cell in a fixed field. The tumor sample can be fixed in formalin paraffin embedded tissue (FFPE).

In some embodiments, the subject who receives the bispecific anti-EGFR/c-Met antibody has a staining intensity score of 1+ or above based on EGFR and/or cMet expression in a tumor sample obtained from the subject as determined by an IHC assay.

In some embodiments, the subject who receives the bispecific anti-EGFR/c-Met antibody has a staining intensity score of 2+ or above based on EGFR and/or cMet expression in a tumor sample obtained from the subject as determined by an IHC assay.

In some embodiments, the subject who receives the bispecific anti-EGFR/c-Met antibody has a staining intensity score of 3+ based on EGFR and/or cMet expression in a tumor sample obtained from the subject as determined by an IHC assay.

In some embodiments, an H score (or histo score) may be assigned to a tumor sample as a semiquantitative approach useful for analyses of immunohistochemical results (Hirsch F R et al., J Clin Oncol 21:3798-3807, 2003; John T et al., Oncogene 28:S14-S23, 2009, incorporated herein by reference in their entireties). In some embodiments, the H score may be based on a predominant staining intensity. In some embodiments, the H score may include the sum of individual H scores for each intensity level seen. As a non-limiting example, the percentage of cells at each staining intensity level may be calculated, and finally, an H score may be assigned using the following exemplary formula: [1×(% cells 1+)+2×(% cells 2+)+3×(% cells 3+)]. The final calculated H score, ranging from 0 to 300, may give more relative weight to higher-intensity membrane staining in a given tumor sample. In some embodiments, the tumor sample may be considered either positive or a negative on the basis of a specific discriminatory threshold.

In some embodiments, a “combined H score” can be generated by adding an H score calculated from the analysis of one biomarker (e.g., EGFR expression) to an H score calculated from the analysis of a second biomarker (e.g., MET expression). Accordingly, the combined H score can have a range of 0 to 600.

### Administration

The bispecific anti-EGFR/c-Met antibody may be administered in a pharmaceutically acceptable carrier. “Carrier” refers to a diluent, adjuvant, excipient, or vehicle with which the antibody of the invention is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used to formulate the bispecific anti-EGFR/c-Met antibody. These solutions are sterile and generally free of particulate matter. They may be sterilized by conventional, well-known sterilization techniques (e.g., filtration). For parenteral administration, the carrier may comprise sterile water and other excipients may be added to increase solubility or preservation. Injectable suspensions or solutions may also be prepared utilizing aqueous carriers along with appropriate additives. For subcutaneous administration, a recombinant human hyaluronidase, such as rHuPH20 (CAS Registry No. 757971-58-7)) may be used. Suitable vehicles and formulations, inclusive of other human proteins, e.g., human serum albumin, are described, for example, in e.g., Remington: The Science and Practice of Pharmacy, 21st Edition, Troy, D. B. ed., Lipincott Williams and Wilkins, Philadelphia, P A 2006, Part 5, Pharmaceutical Manufacturing pp 691-1092. See especially pp. 958-989.

The mode of administration may be any suitable route that delivers the bispecific anti-EGFR-c-Met antibody to the host, such as parenteral administration, e.g., intradermal, intramuscular, intraperitoneal, intravenous or subcutaneous, pulmonary, transmucosal (oral, intranasal, intravaginal, rectal), using a formulation in a tablet, capsule, solution, powder, gel, particle; and contained in a syringe, an implanted device, osmotic pump, cartridge, micropump; or other means appreciated by the skilled artisan, as well known in the art. Site specific administration may be achieved by for example intratumoral, intrarticular, intrabronchial, intraabdominal, intracapsular, intracartilaginous, intracavitary, intracelial, intracerebellar, intracerebroventricular, intracolic, intracervical, intragastric, intrahepatic, intracardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravascular, intravesical, intralesional, vaginal, rectal, buccal, sublingual, intranasal, or transdermal delivery.

In some embodiments, the bispecific anti-EGFR/c-Met antibody is administered intravenously. Exemplary intravenous formulations are disclosed in United States Patent Application Pub. No. US 2022/0064307 A1.

In some embodiments, the bispecific anti-EGFR/c-Met antibody is administered subcutaneously or intradermally to the subject. The bispecific anti-EGFR/c-Met antibody may be administered subcutaneously or intradermally at a dose sufficient to achieve a therapeutic effect in the subject. Exemplary subcutaneous formulations are disclosed in United States Patent Application Pub. No. US 2022/0395573 A1.

In some embodiments, the bispecific anti-EGFR/c-Met antibody is administered at a dose of between about 140 mg to about 1750 mg. In some embodiments, the bispecific anti-EGFR/c-Met antibody is administered at a dose of between about 350 mg to about 2240 mg. In some embodiments, the bispecific anti-EGFR/c-Met antibody is administered at a dose of between about 350 mg to about 1750 mg. In some embodiments, the bispecific anti-EGFR/c-Met antibody is administered at a dose of between about 350 mg to about 3340 mg.

In some embodiments, the bispecific anti-EGFR/c-Met antibody is administered at a dose of about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1010 mg, about 1020 mg, about 1030 mg, about 1040 mg, about 1050 mg, about 1060 mg, about 1070 mg, about 1080 mg, about 1090 mg, about 1100 mg, about 1110 mg, about 1120 mg, about 1130 mg, about 1140 mg, about 1150 mg, about 1160 mg, about 1170 mg, about 1180 mg, about 1190 mg, about 1200 mg, about 1210 mg, about 1220 mg, about 1230 mg, about 1240 mg, about 1250 mg, about 1260 mg, about 1270 mg, about 1280 mg, about 1290 mg, about 1300 mg, about 1310 mg, about 1320 mg, about 1330 mg, about 1340 mg, about 1350 mg, about 1360 mg, about 1370 mg, about 1380 mg, about 1390 mg, about 1400 mg, about 1410 mg, about 1420 mg, about 1430 mg, about 1440 mg, about 1450 mg, about 1460 mg, about 1470 mg, about 1480 mg, about 1490 mg, about 1500 mg, about 1510 mg, about 1520 mg, about 1530 mg, about 1540 mg, about 1550 mg, about 1560 mg, about 1570 mg, about 1580 mg, about 1590 mg, about 1600 mg, about 1610 mg. 1620 mg, about 1630 mg, about 1640 mg, about 1650 mg, about 1660 mg, about 1670 mg, about 1680 mg, about 1690 mg, about 1700 mg, about 1710 mg, about 1720 mg, about 1730 mg, about 1740 mg, about 1750 mg, about 1760 mg, about 1770 mg, about 1780 mg, about 1790 mg, about 1800 mg, about 1810 mg, about 1820 mg, about 1830 mg, about 1840 mg, about 1850 mg, about 1860 mg, about 1870 mg, about 1880 mg, 1890 mg, about 1900 mg, about 1910 mg, about 1920 mg, about 1930 mg, about 1940 mg, about 1950 mg, about 1960 mg, about 1970 mg, about 1980 mg, about 1990 mg, about 2000 mg, about 2010 mg, about 2020 mg, about 2030 mg, about 2040 mg, about 2050 mg, about 2060 mg, about 2070 mg, about 2080 mg, about 2090 mg, about 2100 mg, about 2110 mg, about 2120 mg, about 2130 mg, about 2140 mg, about 2150 mg, about 2160 mg, about 2170 mg about 2180 mg, about 2190 mg, about 2200 mg, about 2210 mg, about 2220 mg, about 2230 mg, about 2240 mg, about 2250 mg, about 2300 mg, about 2400 mg, about 2500 mg, about 2600 mg, about 2700 mg, about 2800 mg, about 2900 mg, about 3000 mg, about 3100 mg, about 3200 mg, about 3300 mg, about 3360 mg, or about 3400 mg.

In some embodiments, the bispecific anti-EGFR/c-Met antibody is administered at a dose of about 350 mg, about 700 mg, about 1050 mg, about 1400 mg, about 1575 mg, about 1600 mg, about 1750 mg, about 2100 mg, about 2240 mg, about 2400 mg, or about 3360 mg. In some embodiments, the bispecific anti-EGFR/c-Met antibody is administered at a dose of about 350 mg. In some embodiments, the bispecific anti-EGFR/c-Met antibody is administered at a dose of about 700 mg. In some embodiments, the bispecific anti-EGFR/c-Met antibody is administered at a dose of about 750 mg. In some embodiments, the bispecific anti-EGFR/c-Met antibody is administered at a dose of about 800 mg. In some embodiments, the bispecific anti-EGFR/c-Met antibody is administered at a dose of about 850 mg. In some embodiments, the bispecific anti-EGFR/c-Met antibody is administered at a dose of about 900 mg. In some embodiments, the bispecific anti-EGFR/c-Met antibody is administered at a dose of about 950 mg. In some embodiments, the bispecific anti-EGFR/c-Met antibody is administered at a dose of about 1000 mg. In some embodiments, the bispecific anti-EGFR/c-Met antibody is administered at a dose of about 1050 mg. In some embodiments, the bispecific anti-EGFR/c-Met antibody is administered at a dose of about 1100 mg. In some embodiments, the bispecific anti-EGFR/c-Met antibody is administered at a dose of about 1150 mg. In some embodiments, the bispecific anti-EGFR/c-Met antibody is administered at a dose of about 1200 mg. In some embodiments, the bispecific anti-EGFR/c-Met antibody is administered at a dose of about 1250 mg. In some embodiments, the bispecific anti-EGFR/c-Met antibody is administered at a dose of about 1300 mg. In some embodiments, the bispecific anti-EGFR/c-Met antibody is administered at a dose of about 1350 mg. In some embodiments, the bispecific anti-EGFR/c-Met antibody is administered at a dose of about 1400 mg. In some embodiments, the bispecific anti-EGFR/c-Met antibody is administered at a dose of about 1575 mg. In some embodiments, the bispecific anti-EGFR/c-Met antibody is administered at a dose of about 1600 mg. In some embodiments, the bispecific anti-EGFR/c-Met antibody is administered at a dose of about 1750 mg. In some embodiments, the bispecific anti-EGFR/c-Met antibody is administered at a dose of about 2100 mg. In some embodiments, the bispecific anti-EGFR/c-Met antibody is administered at a dose of about 2240 mg. In some embodiments, the bispecific anti-EGFR/c-Met antibody is administered at a dose of about 2400 mg. In some embodiments, the bispecific anti-EGFR/c-Met antibody is administered at a dose of about 3360 mg.

In some embodiments, the bispecific anti-EGFR/c-Met antibody is administered is administered at a dose of 1050 mg if the subject has a body weight of less than 80 kg. In some embodiments, the bispecific anti-EGFR/c-Met antibody is administered is administered at a dose of 1050 mg even if the subject has a body weight of greater than or equal to 80 kg.

In some embodiments, the bispecific anti-EGFR/c-Met antibody is administered at a dose of 1400 mg if the subject has a body weight of greater than or equal to 80 kg.

In some embodiments, the bispecific anti-EGFR/c-Met antibody is administered once a week. In some embodiments, the bispecific anti-EGFR/c-Met antibody is administered about 1050 mg once a week. In some embodiments, the bispecific anti-EGFR/c-Met antibody is administered about 1400 mg once a week. In some embodiments, the bispecific anti-EGFR/c-Met antibody is administered about 1600 mg once a week. In some embodiments, the bispecific anti-EGFR/c-Met antibody is administered about 1750 mg once a week. In some embodiments, the bispecific anti-EGFR/c-Met antibody is administered about 2100 mg once a week.

In some embodiments, the bispecific anti-EGFR/c-Met antibody is administered once in two weeks. In some embodiments, the bispecific anti-EGFR/c-Met antibody is administered about 1050 mg once in two weeks. In some embodiments, the bispecific anti-EGFR/c-Met antibody is administered about 1400 mg once in two weeks. In some embodiments, the bispecific anti-EGFR/c-Met antibody is administered about 1600 mg once in two weeks. In some embodiments, the bispecific anti-EGFR/c-Met antibody is administered about 1750 mg once in two weeks. In some embodiments, the bispecific anti-EGFR/c-Met antibody is administered about 2100 mg once in two weeks.

In some embodiments, the bispecific anti-EGFR/c-Met antibody is administered is administered at a dose of 1575 mg if the subject has a body weight of less than 80 kg. In some embodiments, the bispecific anti-EGFR/c-Met antibody is administered at a dose of 2100 mg if the subject has a body weight of greater than or equal to 80 kg.

In some embodiments, the bispecific anti-EGFR/c-Met antibody is administered is administered at a dose of 1600 mg if the subject has a body weight of less than 80 kg. In some embodiments, the bispecific anti-EGFR/c-Met antibody is administered at a dose of 2240 mg if the subject has a body weight of greater than or equal to 80 kg. In some embodiments, the bispecific anti-EGFR/c-Met antibody is administered weekly for the first 4 weeks, and once every 2 weeks thereafter.

In some embodiments, the bispecific anti-EGFR/c-Met antibody is administered is administered at a dose of 2400 mg if the subject has a body weight of less than 80 kg. In some embodiments, the bispecific anti-EGFR/c-Met antibody is administered at a dose of 3360 mg if the subject has a body weight of greater than or equal to 80 kg. In some embodiments, the bispecific anti-EGFR/c-Met antibody is administered weekly for the first 3 weeks, and once every 3 weeks thereafter.

In some embodiments, the bispecific anti-EGFR/c-Met antibody is administered twice a week. In some embodiments, the bispecific anti-EGFR/c-Met antibody is administered once a week. In some embodiments, the bispecific anti-EGFR/c-Met antibody is administered once in two weeks. In some embodiments, the bispecific anti-EGFR/c-Met antibody is administered once in three weeks. In some embodiments, the bispecific anti-EGFR/c-Met antibody is administered once in four weeks.

In some embodiments, the bispecific anti-EGFR/c-Met antibody is administered twice a week, once a week, once in two weeks, once in three weeks or once in four weeks.

In some embodiments, the bispecific anti-EGFR/c-Met antibody is administered once a week for four weeks and once in two weeks thereafter.

In some embodiments, the first dose of the bispecific anti-EGFR/c-Met antibody is administered in two days. As a non-limiting example, the first dose of the bispecific anti-EGFR/c-Met antibody may be split to two days with Day 1 (350 mg) and Day 2 (700 mg if body weight is <80 kg or 1,050 mg if body weight is ≥80 kg).

In some embodiments, the method further comprises administering at least one additional therapeutic to the subject. In some embodiments, the additional therapeutic is a glucocorticosteroid, antihistamine, antipyretic, H₂-antagonist, antiemetic, opiate, or any combination thereof.

In some embodiments, the at least one additional therapeutic is administered prior to the one or more treatment doses.

In some embodiments, the glucocorticosteroid is dexamethasone, beclomethasone, betamethasone, budesonide, cortisone, hydrocortisone, methylprednisolone, prednisolone, prednisone, or triamcinolone. In some embodiments, the glucocorticosteroid is dexamethasone or methylprednisolone. For example, the glucocorticosteroid is dexamethasone (10 mg) or methylprednisolone (40 mg).

The glucocorticosteroid (e.g., dexamethasone, methylprednisolone) may be administered intravenously (IV) approximately 45-60 minutes prior to the administration of the bispecific anti-EGFR/c-Met antibody. Alternatively, the glucocorticosteroid (e.g., dexamethasone, methylprednisolone) may be administered orally 60-90 min prior to the administration of the bispecific anti-EGFR/c-Met antibody.

In some embodiments, the antihistamine is diphenhydramine, brompheniramine, chlorpheniramine, clemastine, cyproheptadine, dexchlorpheniramine dimenhydrinate, doxylamine, hydroxyzine, phenindamine, azelastine, loratadine, cetirizine, desloratadine, or fexofenadine. In some embodiments, the antihistamine is diphenhydramine. For example, the antihistamine may be diphenhydramine (about 25-50 mg) or equivalent.

In some embodiments, the antihistamine medication is administered orally approximately 30-60 prior to administration of the bispecific anti-EGFR/c-Met antibody. In some embodiments, the antihistamine medication is administered intravenously approximately 15 to 30 minutes prior to administration of the bispecific anti-EGFR/c-Met antibody.

In some embodiments, the antipyretic is acetaminophen, ibuprofen, naproxen, ketoprofen, and nimesulide, aspirin, choline salicylate, magnesium salicylate, sodium salicylate, or phenazone (antipyrine). In some embodiments, the antipyretic is acetaminophen. For example, the antipyretic may be acetaminophen (about 650 mg to 1,000 mg) or equivalent.

In some embodiments, the antipyretic medication is administered intravenously approximately 15 to 30 min or orally approximately 30-60 min prior to administration of the bispecific anti-EGFR/c-Met antibody.

In some embodiments, the H₂-antagonist is ranitidine, cimetidine, famotidine, or nizatidine. In some embodiments, the H₂-antagonist is ranitidine. For example, the H₂-antagonist may be ranitidine (about 50 mg) or equivalent.

In some embodiments, the H₂-antagonist medication is administered intravenously approximately 15-30 minutes prior to administration of the bispecific anti-EGFR/c-Met antibody or orally approximately 60 minutes prior to administration of the bispecific anti-EGFR/c-Met antibody.

In some embodiments, the antiemetic is ondansetron, meclizine, dimenhydrinate, prochlorperazine, promethazine, vitamin B6, droperidol, granisetron, metoclopramide, aprepitant, dolasetron, palonosetron, rolapitant. In some embodiments, the antiemetic is ondansetron. For example, the antiemetic may be ondansetron (about 16 mg) or equivalent.

In some embodiments, the antiemetic medication is administered intravenously approximately 15 to 30 minutes prior to administration of the bispecific anti-EGFR/c-Met antibody or orally about 15 to 30 minutes prior to administration of the bispecific anti-EGFR/c-Met antibody.

In some embodiments, the at least one additional therapeutic described herein is administered after the one or more treatment doses. The at least one additional therapeutic may be administered up to 48 hours after the one or more treatment doses if clinically indicated.

As a non-limiting example, a glucocorticosteroid (e.g., dexamethasone (10 mg)), an antihistamine (e.g., diphenhydramine (25-50 mg)), an antipyretic (e.g., acetaminophen (650-1,000 mg)), and/or an opiate (e.g., meperidine (25-100 mg)) may be administered intravenously or orally after the administration of one or more treatment doses of the bispecific anti-EGFR/c-Met antibody if needed. Additionally, an antiemetic medication may be administered intravenously (e.g., ondansetron (8-16 mg)) or orally (e.g., ondansetron (8 mg)) to the subject after the one or more treatment doses if needed.

### Generation of Bispecific Anti-EGFR/c-Met Antibodies

An exemplary bispecific anti-EGFR/c-Met antibody that can be used in the methods of the disclosures is amivantamab. Amivantamab is an IgG1 anti-EGFR/c-Met bispecific antibody described in U.S. Pat. No. 9,593,164, which is incorporated herein by reference in its entirety.

Amivantamab is a low fucose, fully human immunoglobulin G1 (IgG1)-based bispecific antibody directed against the EGFR and MET receptors, shows preclinical activity against tumors with overexpressed wild type EGFR and activation of the MET pathway. Unlike EGFR TKIs, which bind to the intracellular portion of the EGFR, amivantamab targets the extracellular domain of both EGFR and MET. Amivantamab may have at least 3 potential mechanisms of action, including 1) inhibition of ligand-dependent signaling, 2) downregulation of EGFR and MET expression levels, and 3) initiation of antibody-dependent cellular cytotoxicity (ADCC). Amivantamab is produced with low levels of fucosylation, which translates to an enhanced level of ADCC activity. The human FcγIIIa receptor, critical for ADCC, binds low fucose antibodies more tightly and consequently mediates more potent and effective ADCC killing of target cancer cells (Satoh, 2006). It is hypothesized that by targeting the extracellular domain of EGFR and MET, amivantamab can inhibit receptors that display primary resistance to EGFR TKIs (Exon 20 insertion) or have acquired either EGFR resistance mutations (T790M or C797S) or secondary activation of the MET pathway (MET amplification).

Amivantamab is characterized by following amino acid sequences:

### EGFR Binding Arm

>(HCDR1, EGFR binding arm)
SEQ ID NO: 1
TYGMH
>(HCDR2, EGFR binding arm)
SEQ ID NO: 2
VIWDDGSYKYYGDSVKG
>(HCDR3, EGFR binding arm)
SEQ ID NO: 3
DGITMVRGVMKDYFDY
>(LCDR1, EGFR binding arm)
SEQ ID NO: 4
RASQDISSALV
>(LCDR2, EGFR binding arm)
SEQ ID NO: 5
DASSLES
>(LCDR3, EGFR binding arm)
SEQ ID NO: 6
QQFNSYPLT
>(HCDR1, c-Met binding arm)
SEQ ID NO: 7
SYGIS
>(HCDR2, c-Met binding arm)
SEQ ID NO: 8
WISAYNGYTNYAQKLQG
>(HCDR3, c-Met binding arm)
SEQ ID NO: 9
DLRGTNYFDY
>(LCDR1, c-Met binding arm)
SEQ ID NO: 10
RASQGISNWLA
>(LCDR2, c-Met binding arm)
SEQ ID NO: 11
AASSLLS
>(LCDR3, c-Met binding arm)
SEQ ID NO: 12
QQANSFPIT
>(VH, EGFR binding arm)
SEQ ID NO: 13
QVQLVESGGGVVQPGRSLRLSCAASGFTFSTYGMHWVRQAPGKGLEWVA
VIWDDGSYKYYGDSVKGRFTISRDNSKNTLYLQMNSLRAEDTAVYYCAR
DGITMVRGVMKDYFDYWGQGTLVTVSS
>(VL, EGFR binding arm)
SEQ ID NO: 14
AIQLTQSPSSLSASVGDRVTITCRASQDISSALVWYQQKPGKAPKLLIY
DASSLESGVPSRFSGSESGTDFTLTISSLQPEDFATYYCQQFNSYPLTF
GGGTKVEIK
>(VH, c-Met binding arm)
SEQ ID NO: 15
QVQLVQSGAEVKKPGASVKVSCETSGYTFTSYGISWVRQAPGHGLEWMG
WISAYNGYTNYAQKLQGRVTMTTDTSTSTAYMELRSLRSDDTAVYYCAR
DLRGTNYFDYWGQGTLVTVSS
>(VL, c-Met binding arm)
SEQ ID NO: 16
DIQMTQSPSSVSASVGDRVTITCRASQGISNWLAWFQHKPGKAPKLLIY
AASSLLSGVPSRFSGSGSGTDFTLTISSLQPEDFATYYCQQANSFPITF
GQGTRLEIK
>HC1
SEQ ID NO: 17
QVQLVESGGGVVQPGRSLRLSCAASGFTFSTYGMHWVRQAPGKGLEWVA
VIWDDGSYKYYGDSVKGRFTISRDNSKNTLYLQMNSLRAEDTAVYYCAR
DGITMVRGVMKDYFDYWGQGTLVTVSSASTKGPSVFPLAPSSKSTSGGT
AALGCLVKDYFPEPVTVSWNSGALTSGVHTFPAVLQSSGLYSLSSVVTV
PSSSLGTQTYICNVNHKPSNTKVDKRVEPKSCDKTHTCPPCPAPELLGG
PSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESN
GQPENNYKTTPPVLDSDGSFLLYSKLTVDKSRWQQGNVFSCSVMHEALH
NHYTQKSLSLSPGK
>LC1
SEQ ID NO: 18
AIQLTQSPSSLSASVGDRVTITCRASQDISSALVWYQQKPGKAPKLLIY
DASSLESGVPSRFSGSESGTDFTLTISSLQPEDFATYYCQQFNSYPLTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC
>HC2
SEQ ID NO: 19
QVQLVQSGAEVKKPGASVKVSCETSGYTFTSYGISWVRQAPGHGLEWMG
WISAYNGYTNYAQKLQGRVTMTTDTSTSTAYMELRSLRSDDTAVYYCAR
DLRGTNYFDYWGQGTLVTVSSASTKGPSVFPLAPSSKSTSGGTAALGCL
VKDYFPEPVTVSWNSGALTSGVHTFPAVLQSSGLYSLSSVVTVPSSSLG
TQTYICNVNHKPSNTKVDKRVEPKSCDKTHTCPPCPAPELLGGPSVFLF
PPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKG
QPREPQVYTLPPSREEMTKNQVSLTCLVKGFYPSDIAVEWESNGQPENN
YKTTPPVLDSDGSFFLYSRLTVDKSRWQQGNVFSCSVMHEALHNHYTQK
SLSLSPGK
>LC2
SEQ ID NO: 20
DIQMTQSPSSVSASVGDRVTITCRASQGISNWLAWFQHKPGKAPKLLIY
AASSLLSGVPSRFSGSGSGTDFTLTISSLQPEDFATYYCQQANSFPITF
GQGTRLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In some embodiments, the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6; and the second domain comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

In some embodiments, the first domain that specifically binds EGFR comprises a heavy chain variable region (VH) of SEQ ID NO: 13 and a light chain variable region (VL) of SEQ ID NO: 14; and the second domain that specifically binds c-Met comprises the VH of SEQ ID NO: 15 and the VL of SEQ ID NO: 16.

In some embodiments, the bispecific anti-EGFR/c-Met antibody is an IgG1 isotype.

In some embodiments, the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

In one embodiment, the bispecific anti-EGFR/c-Met antibody comprises one or more Fc silencing mutations.

In one embodiment, the one or more Fc silencing mutations decrease affinity to Fcγ receptors.

In one embodiment, the one or more Fc silencing mutations comprise V234A/G237A/P238S/H268A/V309L/A330S/P331S.

In one embodiment, the bispecific anti-EGFR/c-Met antibody comprises a biantennary glycan structure with a fucose content between about 1% to about 15%. Antibodies with reduced fucose content can be made using different methods reported to lead to the successful expression of relatively high defucosylated antibodies bearing the biantennary complex-type of Fc oligosaccharides such as control of culture osmolality (Konno et al., Cytotechnology 64(:249-65, 2012), application of a variant CHO line Lec13 as the host cell line (Shields et al., J Biol Chem 277:26733-26740, 2002), application of a variant CHO line EB66 as the host cell line (Olivier et al., MAbs; 2(4), 2010; Epub ahead of print; PMID:20562582), application of a rat hybridoma cell line YB2/0 as the host cell line (Shinkawa et al., J Biol Chem 278:3466-3473, 2003), introduction of small interfering RNA specifically against the □1,6-fucosyltrasferase (FUT8) gene (Mori et al., Biotechnol Bioeng88:901-908, 2004), or coexpression of β-1,4-N-acetylglucosaminyltransferase III and Golgi α-mannosidase II or a potent alpha-mannosidase I inhibitor, kifunensine (Ferrara et al., J Biol Chem281:5032-5036, 2006, Ferrara et al., Biotechnol Bioeng 93:851-861, 2006; Xhou et al., Biotechnol Bioeng 99:652-65, 2008). In general, lowering fucose content in the glycan of the antibodies potentiates antibody-meidated cellular cytotoxicity (ADCC).

Other bispecific anti-EGFR/c-Met antibodies may also be used in the methods of the disclosure as long as they demonstrate similar characteristics when compared to amivantamab as described in U.S. Pat. No. 9,593,164. Bispecific anti-EGFR/c-Met antibodies that may be used in the methods of the disclosure may also be generated by combining EGFR binding VH/VL domains and c-Met binding VH/VL domains and testing the resulting bispecific antibodies for their characteristics as described in U.S. Pat. No. 9,593,164.

Bispecific anti-EGFR/c-Met antibodies used in the methods of the disclosure may be generated for example using Fab arm exchange (or half molecule exchange) between two monospecific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy chain disulfide bonds in the hinge regions of the parental monospecific antibodies are reduced. The resulting free cysteines of one of the parental monospecific antibodies form an inter heavy-chain disulfide bond with cysteine residues of a second parental monospecific antibody molecule and simultaneously CH3 domains of the parental antibodies release and reform by dissociation-association. The CH3 domains of the Fab arms may be engineered to favor heterodimerization over homodimerization. The resulting product is a bispecific antibody having two Fab arms or half molecules which each bind a distinct epitope, i.e. an epitope on EGFR and an epitope on c-Met. For example, the bispecific antibodies of the invention may be generated using the technology described in Int. Pat. Publ. No. WO2011/131746. Mutations F405L in one heavy chain and K409R in the other heavy chain may be used in case of IgG1 antibodies. For IgG2 antibodies, a wild-type IgG2 and a IgG2 antibody with F405L and R409K substitutions may be used. For IgG4 antibodies, a wild-type IgG4 and a IgG4 antibody with F405L and R409K substitutions may be used. To generate bispecific antibodies, first monospecific bivalent antibody and the second monospecific bivalent antibody are engineered to have the aforementioned mutation in the Fc region, the antibodies are incubated together under reducing conditions sufficient to allow the cysteines in the hinge region to undergo disulfide bond isomerization; thereby generating the bispecific antibody by Fab arm exchange. The incubation conditions may optimally be restored to non-reducing. Exemplary reducing agents that may be used are 2-mercaptoethylamine (2-MEA), dithiothreitol (DTT), dithioerythritol (DTE), glutathione, tris(2-carboxyethyl)phosphine (TCEP), L-cysteine and beta-mercaptoethanol. For example, incubation for at least 90 min at a temperature of at least 20° C. in the presence of at least 25 mM 2-MEA or in the presence of at least 0.5 mM dithiothreitol at a pH of from 5-8, for example at pH of 7.0 or at pH of 7.4 may be used.

Bispecific anti-EGFR/c-Met antibodies used in the methods of the disclosure may also be generated using designs such as the knob-in-hole or knobs-into-holes (Genentech), CrossMAbs (Roche) and the electrostatically-matched (Chugai, Amgen, NovoNordisk, Oncomed), the LUZ-Y (Genentech), the Strand Exchange Engineered Domain body (SEEDbody)(EMD Serono), and the Biclonic (Merus).

In the “knob-in-hole” strategy (see, e.g., Intl. Publ. No. WO 2006/028936) select amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob”. Exemplary CH3 substitution pairs forming a knob and a hole are (expressed as modified position in the first CH3 domain of the first heavy chain/modified position in the second CH3 domain of the second heavy chain): T366Y/F405A, T366W/F405W, F405W/Y407A, T394W/Y407T, T394S/Y407A, T366W/T394S, F405W/T394S and T366W/T366S_L368A_Y407V.

CrossMAb technology, in addition to utilizing the “knob-in-hole” strategy to promoter Fab arm exchange utilizes CH1/CL domain swaps in one half arm to ensure correct light chain pairing of the resulting bispecific antibody (see e.g., U.S. Pat. No. 8,242,247).

Other cross-over strategies may be used to generate full length bispecific antibodies of the invention by exchanging variable or constant, or both domains between the heavy chain and the light chain or within the heavy chain in the bispecific antibodies, either in one or both arms. These exchanges include for example VH-CH1 with VL-CL, VH with VL, CH3 with CL and CH3 with CH1 as described in Int. Patent Publ. Nos. WO2009/080254, WO2009/080251, WO2009/018386 and WO2009/080252.

Other strategies such as promoting heavy chain heterodimerization using electrostatic interactions by substituting positively charged residues at one CH3 surface and negatively charged residues at a second CH3 surface may be used, as described in US Patent Publ. No. US2010/0015133; US Patent Publ. No. US2009/0182127; US Patent Publ. No. US2010/028637 or US Patent Publ. No. US2011/0123532. In other strategies, heterodimerization may be promoted by following substitutions (expressed as modified positions in the first CH3 domain of the first heavy chain/modified position in the second CH3 domain of the second heavy chain): L351Y_F405A_Y407V/T394W, T366I_K392M_T394W/F405A_Y407V. T366L_K392M_T394W/F405A_Y407V, L351Y_Y407A/T366A_K409F, L351Y_Y407A/T366V_K409F, Y407A/T366A_K409F, or T350V_L351Y_F405A_Y407V/T350V_T366L_K392L_T394W as described in U.S. Patent Publ. No. US2012/0149876 or U.S. Patent Publ. No. US2013/0195849.

SEEDbody technology may be utilized to generate bispecific antibodies of the invention. SEEDbodies have, in their constant domains, select IgG residues substituted with IgA residues to promote heterodimerization as described in U.S. Patent No. US20070287170.

Mutations are typically made at the DNA level to a molecule such as the constant domain of the antibody using standard methods.

### Exemplary Embodiments

1. A method of treating gastric cancer in a subject in need thereof, comprising administering to the subject a therapeutically effective amount of a bispecific anti-epidermal growth factor receptor (EGFR)/hepatocyte growth factor receptor (c-Met) antibody.
2. The method of embodiment 1, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
3. The method of embodiment 2, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
4. The method of embodiment 2 or 3, wherein the bispecific anti-EGFR/c-Met antibody is an IgG1 isotype.
5. The method of any one of embodiments 1-4, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
6. The method of any one of embodiments 1-5, wherein the bispecific anti-EGFR/c-Met antibody comprises a biantennary glycan structure with a fucose content of about between 1% to about 15%.
7. The method of any one of embodiments 1-6, wherein the bispecific anti-EGFR/c-Met antibody is administered intravenously or subcutaneously to the subject.
8. The method of embodiment 7, wherein the bispecific anti-EGFR/c-Met antibody is administered at a dose of between about 350 mg to about 2000 mg.
9. The method of embodiment 8, wherein the bispecific anti-EGFR/c-Met antibody is administered at a dose of about 350 mg. 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg. 1950 mg, 2000 mg 2100 mg, 2200 mg, 2240 mg, 2300 mg, 2400 mg. 2500 mg, 2600 mg. 2700 mg, 2800 mg, 2900 mg, 3000 mg, 3100 mg. 3200 mg. 3300 mg, 3360 mg, or 3400 mg.
10. The method of embodiment 9, wherein the bispecific anti-EGFR/c-Met antibody is administered at a dose of 1050 mg.
11. The method of embodiment 9, wherein the bispecific anti-EGFR/c-Met antibody is administered at a dose of 1400 mg.
12. The method of embodiment 9, wherein the bispecific anti-EGFR/c-Met antibody is administered at a dose of 1600 mg.
13. The method of embodiment 9, wherein the bispecific anti-EGFR/c-Met antibody is administered at a dose of 1750 mg.
14. The method of embodiment 9, wherein the bispecific anti-EGFR/c-Met antibody is administered at a dose of 2100 mg.
15. The method of embodiment 9, wherein the bispecific anti-EGFR/c-Met antibody is administered at a dose of 2240 mg.
16. The method of embodiment 9, wherein the bispecific anti-EGFR/c-Met antibody is administered at a dose of 2400 mg.
17. The method of embodiment 9, wherein the bispecific anti-EGFR/c-Met antibody is administered at a dose of 3360 mg.
18. The method of any one of the embodiments 10-15 wherein the bispecific anti-EGFR/c-Met antibody is administered weekly for the first 4 weeks, and once every 2 weeks thereafter.
19. The method of embodiments 16 or 17, wherein the bispecific anti-EGFR/c-Met antibody is administered weekly for the first 3 weeks, and once every 3 weeks thereafter.
20. The method of any one of the embodiments 10-19, wherein the bispecific anti-EGFR/c-Met antibody is administered subcutaneously.
21. The method of embodiment 18, wherein the bispecific anti-EGFR/c-Met antibody is administered intravenously.
22. The method of any one of embodiments 1-6, wherein the bispecific anti-EGFR/c-Met antibody is administered subcutaneously or intradermally to the subject.
23. The method of embodiment 22, wherein the bispecific anti-EGFR/c-Met antibody is administered subcutaneously or intradermally at a dose sufficient to achieve a therapeutic effect in the subject.
24. The method of any one of embodiments 1-23, wherein the bispecific anti-EGFR/c-Met antibody is administered twice a week, once a week, once in two weeks, once in three weeks or once in four weeks.
25. The method of embodiment 24, wherein the bispecific anti-EGFR/c-Met antibody is administered once a week for four weeks and once in two weeks thereafter.
26. The method of embodiment 25, wherein the first dose of the bispecific anti-EGFR/c-Met antibody is administered over two days.
27. The method of any one of embodiments 1-26, wherein one or more cells of the gastric cancer express EGFR and/or cMet.
28. The method of any one of embodiments 1-27, wherein the subject has received a prior treatment.
29. The method of embodiment 28, wherein the prior treatment comprises a chemotherapy, a targeted therapy, an immunotherapy, surgery, radiation therapy, chemoradiation therapy, or a combination thereof.
30. The method of embodiment 29, wherein the chemotherapy comprises a fluoropyrimidine-based chemotherapy, a platinum-based chemotherapy, paclitaxel, irinotecan, or a combination thereof.
31. The method of embodiment 30, wherein the fluoropyrimidine is 5-fluorouracil or capecitabine.
32. The method of embodiment 30, wherein the platinum-based chemotherapy is cisplatin, oxaliplatin, carboplatin, or nedaplatin.
33. The method of embodiment 29, wherein the targeted therapy comprises an anti-HER2 therapy or anti-VEGF/VEGFR therapy.
34. The method of embodiment 33, wherein the anti-HER2 therapy comprises trastuzumab.
35. The method of embodiment 34, wherein the anti-VEGF/VEGFR therapy comprises bevacizumab or ramucirumab.
36. The method of any one of embodiments 1-27, wherein the subject is treatment naïve.
37. The method of any one of embodiments 1-36, wherein the method further comprises administering at least one additional therapeutic to the subject.
38. The method of embodiment 37, wherein the additional therapeutic comprises a glucocorticosteroid, antihistamine, antipyretic, H2-antagonist, antiemetic, opiate, or any combination thereof.
39. The method of any one of embodiments 1-38, wherein the gastric cancer is an advanced or metastatic cancer.
40. The method of any one of embodiments 1-39, wherein the subject is human.
41. A method of treating esophageal cancer in a subject in need thereof, comprising administering to the subject a therapeutically effective amount of a bispecific anti-epidermal growth factor receptor (EGFR)/hepatocyte growth factor receptor (c-Met) antibody.
42. The method of embodiment 41, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.
43. The method of embodiment 42, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.
44. The method of embodiment 42 or 43, wherein the bispecific anti-EGFR/c-Met antibody is an IgG1 isotype.
45. The method of any one of embodiments 41-44, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.
46. The method of any one of embodiments 41-45, wherein the bispecific anti-EGFR/c-Met antibody comprises a biantennary glycan structure with a fucose content of about between 1% to about 15%.
47. The method of any one of embodiments 41-46, wherein the bispecific anti-EGFR/c-Met antibody is administered intravenously or subcutaneously to the subject.
48. The method of embodiment 47, wherein the bispecific anti-EGFR/c-Met antibody is administered at a dose of between about 350 mg to about 2000 mg.
49. The method of embodiment 48, wherein the bispecific anti-EGFR/c-Met antibody is administered at a dose of about 350 mg. 700 mg, about 750 mg, about 800 mg, about 850 mg. 900 mg. 950 mg, 1000 mg. 1050 mg, 1100 mg, 1150 mg, 1200 mg, 1250 mg, 1300 mg. 1350 mg, 1400 mg, 1450 mg. 1500 mg. 1550 mg, 1600 mg, 1650 mg, 1700 mg, 1750 mg, 1800 mg, 1850 mg, 1900 mg, 1950 mg, 2000 mg, or 2100 mg, 2200 mg, 2240 mg, 2300 mg, 2400 mg, 2500 mg. 2600 mg. 2700 mg. 2800 mg, 2900 mg, 3000 mg, 3100 mg, 3200 mg, 3300 mg, 3360 mg, or 3400 mg.
50. The method of embodiment 49, wherein the bispecific anti-EGFR/c-Met antibody is administered at a dose of 1050 mg.
51. The method of embodiment 49, wherein the bispecific anti-EGFR/c-Met antibody is administered at a dose of 1400 mg.
52. The method of embodiment 49, wherein the bispecific anti-EGFR/c-Met antibody is administered at a dose of 1600 mg.
53. The method of embodiment 49, wherein the bispecific anti-EGFR/c-Met antibody is administered at a dose of 1750 mg.
54. The method of embodiment 49, wherein the bispecific anti-EGFR/c-Met antibody is administered at a dose of 2100 mg.
55. The method of embodiment 49, wherein the bispecific anti-EGFR/c-Met antibody is administered at a dose of 2240 mg.
56. The method of embodiment 49, wherein the bispecific anti-EGFR/c-Met antibody is administered at a dose of 2400 mg.
57. The method of embodiment 49, wherein the bispecific anti-EGFR/c-Met antibody is administered at a dose of 3360 mg.
58. The method of any one of the embodiments 50-55 wherein the bispecific anti-EGFR/c-Met antibody is administered weekly for the first 4 weeks, and once every 2 weeks thereafter.
59. The method of embodiments 56 or 57, wherein the bispecific anti-EGFR/c-Met antibody is administered weekly for the first 3 weeks, and once every 3 weeks thereafter.
60. The method of embodiment 58, wherein the bispecific anti-EGFR/c-Met antibody is administered intravenously.
61. The method of embodiment 59, wherein the bispecific anti-EGFR/c-Met antibody is administered subcutaneously.
62. The method of any one of the embodiments 50-59 wherein the bispecific anti-EGFR/c-Met antibody is administered subcutaneously.
63. The method of any one of embodiments 41-57, wherein the bispecific anti-EGFR/c-Met antibody is administered subcutaneously or intradermally to the subject.
64. The method of embodiment 63, wherein the bispecific anti-EGFR/c-Met antibody is administered subcutaneously or intradermally at a dose sufficient to achieve a therapeutic effect in the subject.
65. The method of any one of embodiments 41-64, wherein the bispecific anti-EGFR/c-Met antibody is administered twice a week, once a week, once in two weeks, once in three weeks or once in four weeks.
66. The method of embodiment 65, wherein the bispecific anti-EGFR/c-Met antibody is administered once a week for four weeks and once in two weeks thereafter.
67. The method of embodiment 66, wherein the first dose of the bispecific anti-EGFR/c-Met antibody is administered over two days.
68. The method of any one of embodiments 41-67, wherein one or more cells of the esophageal cancer express EGFR and/or cMet.
69. The method of any one of embodiments 41-68, wherein the subject has received a prior treatment.
70. The method of embodiment 69, wherein the prior treatment comprises a chemotherapy, a targeted therapy, an immunotherapy, surgery, radiation therapy, chemoradiation therapy, or a combination thereof.
71. The method of embodiment 70, wherein the chemotherapy comprises a fluoropyrimidine-based chemotherapy, a platinum-based chemotherapy, paclitaxel, irinotecan, or a combination thereof.
72. The method of embodiment 71, wherein the fluoropyrimidine is 5-fluorouracil or capecitabine.
73. The method of embodiment 71, wherein the platinum-based chemotherapy is cisplatin, oxaliplatin, carboplatin, or nedaplatin.
74. The method of embodiment 70, wherein the targeted therapy comprises an anti-HER2 therapy or anti-VEGF/VEGFR therapy.
75. The method of embodiment 74, wherein the anti-HER2 therapy comprises trastuzumab.
76. The method of embodiment 74, wherein the anti-VEGF/VEGFR therapy comprises bevacizumab or ramucirumab.
77. The method of any one of embodiments 41-68, wherein the subject is treatment naïve.
78. The method of any one of embodiments 41-77, wherein the method further comprises administering at least one additional therapeutic to the subject.
79. The method of embodiment 78, wherein the additional therapeutic is a glucocorticosteroid, antihistamine, antipyretic, H₂-antagonist, antiemetic, opiate, or any combination thereof.
80. The method of any one of embodiments 41-79, wherein the esophageal cancer is an advanced or metastatic cancer.
81. The method of any one of embodiments 41-80, wherein the subject is human.

### EXAMPLES

The following examples are provided to further describe some of the embodiments disclosed herein. The examples are intended to illustrate, not to limit, the disclosed embodiments.

### Example 1. A Phase 2, Open-label Study of Amivantamab in Subjects with Previously Treated Advanced or Metastatic Gastric or Esophageal Cancer Study Rationale

Gastric cancers (GCs) and esophageal cancer (ECs) have been known to express EGFR and cMet and the expression of these proteins have corelated with poor prognosis. Although many agents targeting EGFR are part of standard of care for many tumor types, no anti-EGFR or anti-cMet therapy has been approved in GC or EC. As a bispecific duobody capable of engaging the extracellular domains of both EGFR and cMet receptors, amivantamab has a unique mechanism of action that suggests it has the potential to control EGFR-expressed and/or cMet-expressed GC and EC patients. Amivantamab has demonstrated in vitro and in vivo pre-clinical activities against tumors with the EGFR or cMet amplified GC and EC models (Vijayaraghavan 2020). Furthermore, clinical experience of amivantamab in NSCLC has shown clinical benefit against broad-spectrum of EGFR and cMet aberrations, including EGFR protein overexpression and cMet amplifications.

This study aims to evaluate the clinical activity of amivantamab as a monotherapy in GC (including GEJ cancer) and EC patients who had received at least 2 prior lines (GC/GEJ participants) or 1 prior line (EC participants) of standard therapy. The Phase 2a cohorts aim to initially investigate the anti-tumoractivity of amivantamab in participants with documented expression of either EGFR, cMet, or both as evaluated by immunohistochemistry (IHC). Approximately 30 participants with any expression level of EGFR, cMet, or both proteins are enrolled in each of the Phase 2a cohort. However, based on the priorexperiences investigating anti-EGFR andanti-cMet antibodies in gastroesophageal cancers, at least 20 participants expressing IHC 2+ or higher (defined as participants expressing EGFR IHC 2+ or above and/or cMet IHC 2+ or above) are enrolled. Additional enrollment in Phase 2a cohorts may be allowed to achieve this minimal enrollment. Moreover, at least 10 participants expressing any level of cMet protein are enrolled in each of Phase 2a cohort to better characterize the contribution of cMet in amivantamab activity. If activity is demonstrated in the Phase 2a cohorts, Phase 2a extension cohorts investigating participants without expression of EGFR and cMet, may open for enrollment. The Phase 2b cohorts investigate the clinical activity of amivantamab in selected patient population based on the Phase 2a data.

TABLE 1
Schedule of Activities for Study Procedures/Assessments

| Phase | | | | | | |
|---|---|---|---|---|---|---|
| Screening eening | | Treatment (28 days/cycle) | | | EOT | FU ¹ |
| Study Period | | | | | | |
| Pre- | Full | | | | 30 D After | |
| screening | Screening | Cycle 1 | Cycle 2 | Cycle 3+ | Last Dose | Q12W |

| Day | | | | | | | | | | |
|---|---|---|---|---|---|---|---|---|---|---|
| −28 of C1D1 | 1 | 2 | 8 | 15 | 22 | 1 | 15 | 1 | 15 | — |

Visit Window (Days)

| | | | | | | | | | | |
|---|---|---|---|---|---|---|---|---|---|---|
| — | — | ±1 | ±1 | ±1 | ±1 | ±1 | ±3 | ±3 | +7 | ±14 |

STUDY PROCEDURES
Treatment cycles are 28 days in duration. Assessments during in-clinic dosing days are performed prior to administration
of study treatment unless otherwise stated. Starting with Cycle 2 Day 1: if a dose interruption or missed dose leads to a
dose delay, the sampling schedule (except disease assessments) is delayed accordingly to ensure sampling relative to
amivantamab dose administration. In Follow-up phase, data are collected until the end of study unless the participant has
died, is lost to follow-up, or has withdrawn consent.
EFFICACY ASESSMENTS

| CT/MRI tumor | X | 6 wks (+1 wk) for first assessment, then every 6 wks | |
|---|---|---|---|
| imaging^{h} | | (±1 wk) for first 12 months then every 12 wks (±1 wk) | |
| Brain imaging | X | As clinically indicated | |
| Survival and | | | X |
| Subsequent anticancer | | | |
| therapies | | | |

Abbreviations:
AE = adverseevent;
C = Cycle;
ctDNA = circulating tumor DNA;
CT = computerized tomography;
cMet = tyrosine-protein kinase mesenchymal-epithelial transition;
D = Day;
ECG = electrocardiogram;
ECOG = Eastern Cooperative Oncology Group;
EGFR = epidermal growth factor receptor;
EoT = end-of-treatment;
FU = follow-up;
HBV = hepatitis B virus;
HCV = hepatitis C virus;
HIV = human immunodeficiency virus;
h = hour;
ICF = informed consent form;
IHC = Immunohistochemistry;
IV = intravenous;
MRI = magneticresonance imaging;
Q12W = every 12 weeks;
wk(s) = week(s)

### Benefit-risk Assessment

### Risks for Study Participation

The safety and tolerability of amivantamab monotherapy was shown in the Phase 1 Study 61186372EDI1001 in NSCLC patients. Amivantamab was generally well tolerated without any occurrence of dose limiting toxicities (DLTs). However, given that the available clinical data are limited to NSCLC participants, unforeseen safety risks associated with the study treatments are possible in GC or EC participants. This study protocol includes the following elements to mitigate risks for study participants:
Participants are monitored closely for safety throughout the study (refer to Safety Assessments Section), per the scheduled assessments outlined in the Schedule of Activities (Table 1).
Dose modification guidance is provided to manage toxicities that occur during the study (refer to Dose Delay Guidance Section and Dose Modification Guidance Section), including specific guidance for infusion-related reactions (IRRs), rash, interstitial lung disease (ILD), liver test abnormalities, or paronychia.

Amivantamab is generally safe and well tolerated based on the data mentioned above.

### Benefits for Study Participation

Although many agents targeting EGFR have been approved and are part of standard of care for many tumor types, no anti-EGFR or anti-cMet therapy has been approved in GC or EC. Amivantamab has demonstrated significant activity as monotherapy for the treatment of NSCLC, receiving Breakthrough Therapy Designation by the US FDA and China Center for Drug Evaluation, based on an overall response rate of 41% in subjects with EGFR Exon20ins disease, after prior treatment with platinum-based chemotherapy. Consistent with the unique mechanism of action of amivantamab, activity was observed in subjects with diverse EGFR mutations, as well as in subjects with amplification of MET and overexpressed EGFR.

It is anticipated that using this therapeutic targeted approach with amivantamab as a single agent in either advanced GC (including GEJ cancer) or EC participants may provide benefit to these participants.

### Benefit-risk Assessment for Study Participation

Considering the measures taken to minimize risk to participants of this study (refer to Risks for Study Participation Section), the potential risks of amivantamab are justified by the anticipated benefits that may be afforded to participants with advanced GC or EC (refer to Benefits for Study Participation Section).

TABLE 2
Objectives and Endpoints

| | |
|---|---|
| Objectives | Endpoints |

Primary

| | |
|---|---|
| To investigate the activity of amivantamab | Objective response rate, as determined |
| in gastric cancer (GC) and esophageal | by investigator, according to the |
| cancer (EC) participants (Phase 2a) | Response Criteria in Solid Tumors |
| To characterize the preliminary antitumor | (RECIST) Version 1.1. |
| activity of amivantamab in selected GC | |
| and EC population (Phase 2b) | |

Secondary

| | |
|---|---|
| To assess additional measures of clinical | Disease control rate (DCR), duration of |
| benefits with amivantamab | response (DoR), progression-free |
| | survival (PFS), and overall survival |
| | (OS; Phase 2b only). |
| To confirm the safety of amivantamab in | Adverse events defined by the National |
| participants with gastric oresophageal | Cancer Institute Common Terminology |
| cancers (ECs) | Criteria for Adverse Events (NCI- |
| | CTCAE) Version 5.0 in participants |
| | treated with amivantamab |
| To assess the pharmacokinetics (PK) | Serum PK parameters including but not |
| and immunogenicity of amivantamab | limited to maximum serum concentration |
| following multiple intravenous dose | (Cₘₐₓ), time to reach the maximum serum |
| administrations | concentration (Tₘₐₓ), area under the curve |
| | (AUC)_{(t1-t2),} AUCₜₐᵤ, plasma/serum |
| | concentration immediately prior the next |
| | study treatment administration (C_{trough}), |
| | and accumulation ratio; incidence of |
| | anti-amivantamab antibodies |

Exploratory
Explore biomarkers predictive of clinical response and resistance to amivantamab
in blood and tumor tissue
Explore the relationship between serum PK and pharmacodynamic (PD) markers
(e.g., soluble EGFR and cMet)
Determine whether amivantamab can impact the anti-tumor immune response
or microenvironment

### Hypothesis

Amivantamab is expected to exhibit anti-tumor activity in participants with GC or EC expressing any level of EGFR, cMET, or both proteins.

### Study Design

### Overall Design

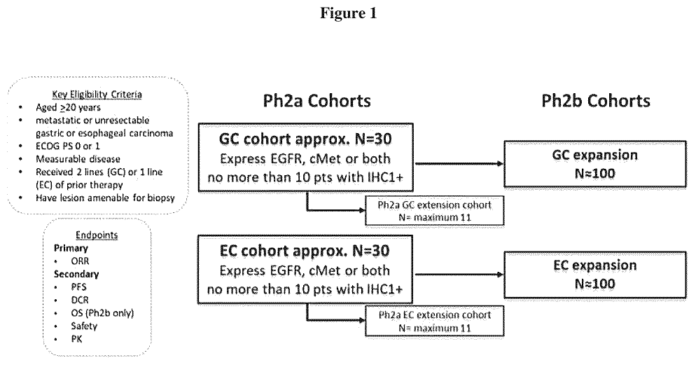

This is an open-label, multicenter, multi-arm Phase 2 interventional study in participants with previously treated advanced or unresectable GC or EC who are 20 years or older (or the legal age of consent in the jurisdiction in which the study is taking place). Japanese participants with gastric/GEJ or EC who express varying degrees of EGFR, cMet, or both as determined by IHC locally or centrally are enrolled in the GC cohort or EC cohort. If activity is demonstrated in the Phase 2a cohorts, Phase 2a extension cohorts investigating participants without expression of EGFR and cMet, may open forenrollment. If activity is observed within the Phase 2a cohorts, the corresponding Phase 2b GC or EC expansion cohorts may be initiated to evaluate the antitumor activity of amivantamab in selected GC and EC participants based upon the prospective assessment of IHC during Phase 2a (FIG. 1).

A maximum of approximately 282 participants are enrolled in the combined Phase 2a and Phase 2b populations, in the event the efficacy observed in both Phase 2a cohorts warrants full enrollment in their respective Phase 2b cohorts. Approximately, 30 response evaluable participants are enrolled in each of Phase 2a GC and EC arm. However, based on the prior experiences investigating anti-EGFR and anti-cMet antibodies in gastroesophageal cancers, at least 20 participants expressing IHC 2+ or higher (defined as participants expressing EGFR IHC 2+ or above or cMet IHC 2+ or above) are enrolled. Moreover, at least 10 participants expressing any level of cMet protein (IHC1+ or above) are enrolled in each Phase 2a cohort to better characterize the contribution of cMet in amivantamab activity. Additional enrollment may be allowed in the Phase 2a cohorts to achieve this minimal enrollment, if these criteria aren't met in the initial 30 evaluable subjects. If activity is demonstrated in the Phase 2a cohorts, Phase 2a expansion cohorts investigating a maximum of 11 participants without any expression of EGFR and cMet, may open for enrollment. The Phase 2b GC expansion or EC expansion cohorts evaluate the antitumor activity of amivantamab in GC and EC patients, using biomarker selection based upon Phase 2a results. If activated, approximately 100 participants are enrolled in each of the Phase 2b cohorts.

The study includes a screening phase (Screening Phase (Pre- and Full Screening) Section), a treatment phase (Treatment Phase Section), and a follow-up phase (Follow-up Phase (Applicable to Phase 2b Only) Section).

### Screening Phase (Pre- and Full Screening)

During the full screening period, participants are evaluated for eligibility for study participation. Participants complete all screening procedures within 28 days of C1D1. Pre-treatment biopsy is collected for all participants in Phase 2a and Phase 2b. The screening period can be extended by 14 days if reporting of central tumor IHC results are not completed within the 28-day screening period. However, all other assessments still meet timing criteria relative to C1D1 or are repeated.

An optional pre-screening period is offered to facilitate molecular characterization of archived tumor biopsy sample. The participant may submit archival sample before completion of the previous therapy.

### Treatment Phase

The treatment phase for a participant begins on C1D1 and continue as 28-day cycles until the end-of-treatment (EOT) visit, approximately 30 days after discontinuation of study treatment. This study is conducted in an outpatient setting. However, in-hospital observation, from C1D1 until C1D8 is permitted in the Phase 2a (including Phase 2a extension cohorts) to allow close monitoring. Study treatment continues until documented clinical or radiographic (RECIST Version 1.1) disease progression or until the participant meets another criterion for discontinuation of study treatment.

Disease assessments occur as close as possible to the start of treatment (baseline screening scans), 6 weeks (+1 week) after the first dose of study treatment, then every 6 weeks (±1 week) for the first 12 months and then every 12 weeks (±1 week) until objective radiographic disease progression or withdrawal of consent.

At each study visit during the treatment phase, participants undergo safety evaluations, including physical examinations and assessment of adverse events (AEs), vital signs, concomitant medication usage, and clinical laboratory parameters. Participants also have blood samples drawn for assessment of PK and immunogenicity parameters and for biomarker evaluations, at selected visits. Post-treatment biopsy, circulating tumor DNA (ctDNA), and biomarker at C2D15 as well as per the Schedule of Activities Section are collected on C2D15 (+1 week) from all participants in Phase 2a.

### Follow-up Phase (Applicable to Phase 2b Only)

Participants who discontinue study treatment are followed for subsequent therapy, disease status (applicable only if participants discontinuing treatment due to reasons other than progressive disease, to confirm disease progression date), and survival in the follow-up phase. This phase starts from the EOT visit assessment is done every 12 weeks (±14 days) after the last dose of study treatment or disease progression (whichever occurs first) and continues until the end of study, death, lost to follow-up, or withdrawal of consent from participation in the study, whichever comes first.

A diagram of the study design is provided in FIG. 1.

### Justification for Dose

Amivantamab was generally well tolerated in a Phase 1 study (Study 61186372EDI1001) up to the dose of 1750 mg, with no dose limiting toxicities reported during dose escalation and no maximum tolerated dose identified in lung cancer participants. Based on the totality of exposure, safety, and efficacy data, the recommended Phase 2 dose was determined to be 1050 mg for body weight <80 kg and 1400 mg for body weight ≥80 kg, administered by IV infusion in 28-day cycles: once weekly in Cycle 1 (with a split dose on Days 1-2), and then every 2 weeks in subsequent cycles. The recommended Phase 2 dose achieved a complete soluble target saturation throughout dosing for the EGFR and cMet in the lung cancer participants. The observed safety profile of amivantamab is consistent with EGFR and cMet inhibition and majority of treatment emergent adverse events (TEAEs) were Grade 1 to 2 in severity. Therefore, administering the same dosing regimen to GC or EC participants is considered appropriate.

### Study Population

Screening for eligible participants is performed within 28 days before administration of the study treatment.

The inclusion and exclusion criteria for enrolling participants in this study are described below.

### Inclusion Criteria

Each potential participant satisfies all of the following criteria to be enrolled in the study.
1. Participant is ≥20 years of age (or the legal age of consent in the jurisdiction in which the study is taking place).
2. Participant tumor express either EGFR, cMet, or both as determined by either local or central IHC assay (IHC 1+ or above). A copy of the de-identified pathology report for IHC analysis is submitted during the screening period if local IHC test was used for the eligibility determination. Phase 2a extension cohorts: Participant tumor lacks expression of EGFR and MET as determined by either local or central IHC assessment. A copy of the de-identified pathology report for IHC analysis is submitted during the screening period if local IHC test was used for the eligibility determination.
3. Participant has histologically or cytologically confirmed gastric (including GEJ) or EC that is locally advanced, unresectable, or metastatic, and not eligible for curative treatment.

Gastric or GEJ Cancer Only: Participant is refractory or ineligible to at least 2 prior lines of standard of care systemic therapy. Prior therapies include fluoropyrimidine- and platinum-based chemotherapy. Participants with known HER2 expression have had HER2-targeting therapy as part of the prior therapy. In case of progression within 24 weeks of prior adjuvant or neoadjuvant chemotherapy, this therapy is considered as 1 prior line of systemic therapy for the purpose of meeting the eligibility criteria.

Esophageal Cancer Only: (a) Participant is refractory or intolerant to at least 1 prior line of systemic therapy. Prior therapies include fluoropyrimidine-, and platinum-based chemotherapy (including chemoradiation therapy given as stage IV setting). (b) Participant who underwent a radical resection in conjunction with chemotherapy including neo-adjuvant/adjuvant therapy and chemoradiation (including participants who underwent chemoradiation, if residual tumor exists, followed by salvage surgery) whose recurrence was confirmed by imaging within 24 weeks after the last dose of chemotherapy are considered as having received 1 line of prior systemic therapy for the purpose of meeting the eligibility criteria. If prior combination therapy discontinued due to an AE, and then one of the agents continued, this is considered to be “1 prior line” and not “2 prior lines.” The change in dosage form (IV administration, oral administration) or dose reduction without progression is considered to be “1 prior line” and not “2 prior lines.”
4. Participant has measurable disease according to RECIST Version 1.1. If only one measurable lesion exists, it may be used for the screening biopsy if the baseline tumor assessment scans are performed ≥7 days after the biopsy.
5. Participant has Eastern Cooperative Oncology Group (ECOG) performance status 0 or 1 (Eastern Cooperative Oncology Group (ECOG) Performance Status).
6. Participant has adequate organ and bone marrow function as follows, without history of red blood cell transfusion or platelet transfusion within 7 days prior to the date of the laboratory test.
a. Hemoglobin ≥8 g/dL
b. Absolute neutrophil count ≥1500/mm³, without use of granulocyte colony stimulating factor (G-CSF) within 10 days prior to the date of the test
c. Platelets ≥75,000/mm³
d. Alanine aminotransferase (ALT) and aspartate aminotransferase (AST) ≤3×upper limit of normal (ULN). If liver metastases are present, ≤5×ULN
e. Total bilirubin≤1.5×ULN (participants with Gilbert's syndrome can enroll if direct bilirubin is within normal limits)
f. Calculated or measured glomerular filtration rate ≥40 mL/min using the Modified Diet in Renal Disease (MDRD) equation (refer to MDRD formula for eGFR)
7. Participant has a tumor lesion amenable for biopsy and agree to protocol-defined mandatory biopsies.

### Exclusion Criteria

Any potential participant who meets any of the following criteria are excluded from participating in the study.
1. Participant has an uncontrolled illness, including but not limited to the following:
a. Diabetes
b. Ongoing or active bacterial infection (includes infection requiring treatment with antimicrobial therapy [participants are required to complete antibiotics 1 week before enrollment]), symptomatic viral infection, or any other clinically significant infection
c. Active bleeding diathesis
d. Psychiatric illness/social situation that would limit compliance with study requirements
2. Participant has had prior chemotherapy, targeted cancer therapy, immunotherapy, or treatment with an investigational anticancer agent within 2 weeks or 4 half-lives whichever is longer or had radiation therapy within 4 weeks before the first administration of study treatment. For agents with long half-lives, the maximum required time since last dose is 28 days. Toxicities from previous anticancer therapies should have resolved to baseline levels or to Grade 1 or less, (except for alopecia or post-radiation skin changes [any grade], Grade≤2 peripheral neuropathy, and Grade≤2 hypothyroidism stable on hormone replacement).
3. Participant has untreated brain metastases (a participant with definitively, locally treated metastases who is clinically stable, asymptomatic, and of corticosteroid treatment for at least 2 weeks prior to the first administration of study treatment is eligible), history of leptomeningeal disease or spinal cord compression that has not been treated definitively with surgery or radiation. If brain metastases are diagnosed on screening imaging, the participant may be rescreened for eligibility after definitive treatment.
4. Participant has a history of (non-infectious) ILD/pneumonitis that required steroids, or has current ILD/pneumonitis, or where suspected ILD/pneumonitis cannot be ruled out by imaging at screening. Esophageal cancer participants with history of completely resolved radiation pneumonitis (defined as radiographically stable for 3 months prior to enrollment without need of any treatment) may be enrolled.
5. Participant has an active malignancy (i.e., progressing or requiring treatment change in the last 12 months) other than the disease being treated under study. The only allowed exceptions are:
a. Non-muscle invasive bladder cancer treated within the last 24 months that is considered completely cured.
b. Skin cancer (non-melanoma or melanoma) treated within the last 24 months that is considered completely cured.
c. Non-invasive cervical cancer treated within the last 24 months that is considered completely cured.
d. Localized prostate cancer (NOMO):
   With a Gleason score of 6, treated within the last 24 months or untreated and under surveillance,
   With a Gleason score of 3+4 that has been treated more than 6 months prior to full study screening and considered to have a very low risk of recurrence,
   Or history of localized prostate cancer and receiving androgen deprivation therapy and considered to have a very low risk of recurrence.
e. Breast cancer:
   adequately treated lobular carcinoma in situ or ductal carcinoma in situ,
   or history of localized breast cancer and receiving antihormonal agents and considered to have a very low risk of recurrence.
f. Malignancy that is considered cured with minimal risk of recurrence.
g. Phase 2a participants only: Participants with a second malignancy may enroll if any systematic treatment is considered not to become necessary for at least 6 months after the enrollment.
6. Participant has a history of clinically significant cardiovascular disease including, but not limited to:
a. Diagnosis of deep vein thrombosis or pulmonary embolism within 4 weeks prior to the first dose of study treatment or any of the following within 6 months prior to the first dose of study treatment: myocardial infarction, unstable angina, stroke, transient ischemic attack, coronary/peripheral artery bypass graft, or any acute coronary syndrome. Clinically non-significant thrombosis, such as non-obstructive catheter-associated clots, are not exclusionary.
b. Prolonged QTcF interval >480 msec or clinically significant cardiac arrhythmia or electrophysiologic disease (eg, placement of implantable cardioverter defibrillator or atrial fibrillation with uncontrolled rate). Participants with cardiac pacemakers who are clinically stable are eligible.
c. Uncontrolled (persistent) hypertension: systolic blood pressure >180 mm Hg; diastolic blood pressure >100 mm Hg.
d. Congestive heart failure defined as New York Heart Association (NYHA) class III-IV or Hospitalization for congestive heart failure (any NYHA class) (New York Heart Association Criteria) within 6 months of study enrollment.
e. Pericarditis/clinically significant pericardial effusion.
f. Myocarditis.
7. Participant has known allergies, hypersensitivity, or intolerance to excipients of amivantamab.
8. Participant has received study treatment (including investigational vaccines) within 6 weeks before enrollment except for the anticancer therapy described in the Exclusion Criterion No. 2.
9. Participant has, or is expected to have, any of the following:
a. An invasive operative procedure with entry into a body cavity, within 4 weeks or without complete recovery before C1D1. Thoracentesis, if needed, and percutaneous biopsy for baseline tumor tissue sample may be done less than 4 weeks prior to C1D1, as long as the participant has adequately recovered from the procedure prior to the first dose of study treatment in the clinical judgement of the investigator
b. Significant traumatic injury within 3 weeks before the start of C1D1 (unless all wounds are fully healed prior to Day 1)
c. Expected major surgery while the investigational agent is being administered or within 6 months after the last dose of study treatment
10. Participant has at screening:
a. Positive hepatitis B (hepatitis B virus [HBV]) surface antigen (HBsAg). Participants with a prior history of HBV demonstrated by positive hepatitis B core antibody are eligible if they have at screening 1) a negative HBsAg and 2) an HBV DNA (viral load) below the lower limit of quantification, per local testing. Participants with a positive HBsAg due to recent vaccination are eligible if HBV DNA (viral load) is below the lower limit of quantification, per local testing.
b. Positive hepatitis C antibody (anti-HCV [hepatitis C virus]). Participants with a prior history of HCV, who have completed antiviral treatment and have subsequently documented HCV RNA below the lower limit of quantification per local testing are eligible.
c. Other clinically active infectious or non-infectious liver disease
11. Participant is known to be positive for human immunodeficiency virus (HIV), with 1 or more of the following:
a. Not receiving highly active antiretroviral therapy (ART)
b. Had a change in ART within 6 months of the start of screening
c. Receiving ART that may interfere with study treatment
d. CD4 count <350 at screening
e. Acquired immunodeficiency syndrome (AIDS)-defining opportunistic infection within 6 months of start of screening. Not agreeing to start ART and be on ART >4 weeks plus having HIV viral load <400 copies/mL at end of 4-week period (to ensure ART is tolerated and HIV controlled).
12. Participant has received prior EGFR or cMet-directed therapies.

### Activity

Participants agree to use sun protective measures (such as a hat, sunglasses, protective clothing, sunscreen), limit prolonged exposure to natural sunlight, and avoid artificial sunlight (tanning beds or phototherapy) from baseline until the last dose of study treatment. Use broad-spectrum sunscreen (containing titanium dioxide or zinc oxide) with a skin protection factor ≥15.

### Study Treatment Administered

Amivantamab is supplied for this study in a glass vial containing 350 mg/vial with concentration of 50 mg/mL in a 7 mL vial. The IV infusion is prepared at the site in 250 mL of diluent.

The initial dosage of amivantamab is based on the participant's body weight at screening: 1,050 mg (if body weight is <80 kg) or 1,400 mg (if body weight is ≥80 kg). s Amivantamab is administered as an IV infusion in 28-day cycles as follows:
Cycle 1: Once weekly (with the first dose split over Day 1 [350 mg] and Day 2 [700 mg if body weight is <80 kg or 1,050 mg if body weight is ≥80 kg]).
Cycles 2+: Days 1 and 15 of each cycle.

Amivantamab may be administered at a higher dose based on body weight: 1750 mg for body weight <80 kg and 2100 mg for body weight >=80 kg as IV infusion in 28-day cycles as follows:
Cycle 1: Once weekly (with the first dose split over Day 1 [350 mg] and Day 2 [1400 mg if body weight is <80 kg or 1,750 mg if body weight is ≥80 kg]).
Cycles 2+: Days 1 and 15 of each cycle.

Amivantamab is administered intravenously using an escalating infusion rate regimen. The product is infused via a peripheral vein for all Cycle 1 doses; infusion via central line is allowed for subsequent dosing starting with the C2D1 dose.

Amivantamab is administered according to clinical protocol. Additional guidance is provided below:
On Day 1 of each cycle, results of hematology and chemistry laboratory assessments are reviewed before administering study treatment.
Amivantamab is diluted prior to infusion
Amivantamab is not mixed or diluted with other drugs.
Amivantamab is not administered as an IV push or bolus.

Dose and administration schedule may be adjusted during this study.

### Preparation/Handling/Storage/Accountability

### Preparation/Handling/Storage

All study treatment are stored at controlled temperatures according to the requirements on the label. Amivantamab is protected from light prior to use.

### Measures to Minimize Bias: Randomization and Blinding

### Treatment Allocation

Procedures for Randomization: Randomization is not used in this study. Participants are assigned to treatment in the order in which they qualify for this study.

### Blinding

As this is an open-label study, blinding procedures are not applicable.

### Dose Delay Guidance

In instances where treatment delay is indicated, treatment with amivantamab may be delayed until recovery of toxicity to a level allowing continuation of therapy. A participant for whom treatment was delayed is assessed at least weekly to ensure adequate supportive care is being administered and to assess for improvement of toxicity. For majority of clinically significant toxicities withholding doses and dose modifications may be made as per the guidelines described below (Dose Modification Guidance Section).

The following sections provide additional guidance for the prevention, monitoring, and management of toxicities that have been reported with amivantamab.

### Infusion-related Reactions

Infusion-related reactions have been commonly observed during treatment with amivantamab, predominantly with the first exposure on C1D1, and typically within the first 90 minutes of the infusion. The majority of IRRs are Grade 1 or 2. The guidelines described here are related to the safe administration of amivantamab during initial dosing.

During the amivantamab infusions, participants are clinically monitored at regular intervals (including an assessment prior to the start of infusion). The monitoring includes heart rate, blood pressure, temperature, respiratory rate, and oxygen saturation measurements.

### Amivantamab Pre-infusion and Post-infusion Medications

### Amivantamab Pre-infusion Medications

Required and optional amivantamab pre-infusion medications for IRRs are summarized in Table 3.

TABLE 3
Amivantamab Pre-infusion Medications

| | | | | |
|---|---|---|---|---|
| | | | Recommended | |
| | | Route of | Dosing Window | |
| Medication | Dose | Administration | Before Infusion | Cycle/Day |

Required Pre-infusion Medications^{a, b}

| | | | | |
|---|---|---|---|---|
| Glucocorticoid | Dexamethasone (10 mg) | IV | 45 to 60 minutes | C1D1 |
| | or Methylprednisolone | | | C1D2 |
| | (40 mg) | | | |
| Antihistamine | Diphenhydramine (25 | IV | 15 to 30 minutes | All |
| | to 50 mg) or equivalent | Oral | 30 to 60 minutes | |
| Antipyretic | Paracetamol | IV | 15 to 30 minutes | All |
| | (acetaminophen 650 to | Oral | 30 to 60 minutes | |
| | 1,000 mg) or equivalent | | | |

Optional Pre-infusion Medications^{a}

| | | | | |
|---|---|---|---|---|
| Glucocorticoid^{c} | Dexamethasone (10 mg) | IV | 45 to 60 minutes | C1D8 and |
| | Methylprednisolone | Oral | 60 to 90 minutes | beyond |
| | (40 mg) | | | |
| H₂-antagonist | Ranitidine (50 mg) | IV | 15 to 30 minutes | Any |
| | or equivalent | | | |
| Antiemetic | Ondansetron (16 mg) | IV | 15 to 30 minutes | Any |
| | or equivalent | | | |
| | Ondansetron (8 mg) | Oral | 15 to 30 minutes | |
| | or equivalent | | | |

C = Cycle; D = Day X; IV = intravenous.
^{a}If a medication noted in this table is not locally available, a similar medication and dose may be substituted and administered per local guidelines.
^{b}Participants for whom required medications are contraindicated may explore alternative medications with their study physician. If alternative medications are not suitable for the intent above, participants are not required to take the corresponding medication.
^{c}Beginning with C1D8, optional predose steroids may be administered if clinically indicated for participants who experienced an infusion-related reaction on C1D1 or C1D2.

### Amivantamab Post-infusion Medications

Optional amivantamab post-infusion medications may be prescribed and continued for up to 48 hours after the infusion if clinically indicated as described in Table 4.

TABLE 4
Amivantamab Post-infusion Medications

| | | | | |
|---|---|---|---|---|
| | | Route of | Administration | Cycle/ |
| Medication | Dose | Administration | Instructions | Day |

Optional Post-infusion Medications^{a}

| | | | | |
|---|---|---|---|---|
| Glucocorticoid | Dexamethasone (10 mg) or | IV or Oral | As clinically | Any |
| | comparable corticosteroid | | indicated | |
| Antihistamine | Diphenhydramine (25-50 mg) | IV or Oral | As clinically | Any |
| | or equivalent | | indicated | |
| Antipyretic | Paracetamol (acetaminophen) | IV or Oral | As clinically | Any |
| | (650-1,000 mg) | | indicated | |
| Opiates | Meperidine (25-100 mg) | IV or Oral | As clinically | Any |
| | | | indicated | |
| Antiemetic | Ondansetron (8-16 mg) or | IV | As clinically | Any |
| | equivalent; long or short | | indicated | |
| | acting agents | | | |
| | Ondansetron (8 mg) or | Oral | | |
| | equivalent; long or short | | | |
| | acting agents | | | |

IV = intravenous.
^{a}Optional medications can be used prophylactically as clinically indicated. If a medication noted in this table is not locally available, a similar medication and dose may be substituted and administered per local guidelines

### Prohibited or Restricted Medications and Therapies

The following concomitant medications and therapies are not used during the study.
Any chemotherapy, systemic anticancer therapy, or experimental therapy (other than study treatments). Hormonal treatments as specified in exclusion 5 are allowed.
Radiotherapy to tumor lesions being assessed for tumor response prior to radiographic progression
Use of live or live attenuated vaccines is prohibited
Due to the potential for hypomagnesemia associated with EGFR inhibitors, concomitant medications that may decrease serum magnesium are avoided if possible.

### Study Assessments and Procedures

### Overview

The Schedule of Activities Section summarizes the frequency and timing of the measurements applicable to this study.

The total blood volume collected for the study is approximately 25 mL (screening), 105 mL (Cycle 1), 75 mL (Cycle 2), and 30 mL (for each cycle beyond Cycle 3 and EOT).

Repeat or unscheduled samples may be taken for safety reasons or for technical issues with the samples.

### Efficacy and Immunogenicity Assessments

### Disease Assessments

Disease assessments are performed as described in the Schedule of Activities (Table 1) regardless of any dose modifications. More frequent radiologic assessments are allowed if clinically indicated.

Computerized tomography scan of the chest (including the supraclavicular region), abdomen, pelvis, and any other disease location(s), if clinically indicated, is performed with an IV contrast agent. Participants not able to undergo CT scans with IV contrast (e.g., due to allergy or renal insufficiency) may have non-contrast CT of the thorax and MRI of the abdomen and pelvis with IV contrast at baseline and during the study. Identical methodology is used for disease assessment at baseline and throughout the course of the study to characterize each identified and reported lesion to document disease status. Techniques other than CT or MRI may be used based upon local standard of care, and RECIST Version 1.1 guidelines for the use of these alternative techniques.

The baseline disease assessments are performed as close as possible to the start of treatment, but no more than 28 days prior to the first dose. Subsequent assessments are performed at 6 weeks (+1 week) after initiation of study treatment administration, then every 6 weeks (±1 week) for the first 12 months and then every 12 weeks (±1 week) until objective radiographic disease progression. Timing for each disease assessment is relative to the first dose of study treatment administration, regardless of dose modifications, and continues until disease progression. Any other site at which new disease is suspected is also imaged.

If a participant achieves partial response (PR) or complete response (CR), the response is confirmed after, but as close to, 4 weeks of entering PR or CR instead of predefined 6 weeks.

If it is unclear as to whether progression has occurred, particularly with response to nontarget lesions or the appearance of a new lesion, treatment is continued until the next scheduled assessment (or sooner if clinically indicated) and reassess the participant's status. If the repeated scans confirm progression, then the date of the initial scan is declared as the date of progression. To achieve “unequivocal progression” on the basis of non target lesions, there must be an overall substantial worsening in nontarget lesions such that, even in the presence of stable disease or PR in target lesions, the overall tumor burden has increased sufficiently to merit discontinuation of therapy. A modest “increase” in size of 1 or more nontarget lesions is usually not sufficient to qualify for unequivocal progression. If symptomatic deterioration (on the basis of global deterioration of health status) is recorded as the basis for determining disease progression, then the clinical findings used to make the determination are specified. Radiographic progression is documented even after discontinuation of treatment for symptomatic deterioration, but prior to subsequent therapy, if possible. For participants who discontinue study treatment due to toxicity or a reason other than objective progressive disease, tumor assessments is continued per schedule until radiographic progressive disease is documented.

Participants have brain MRI scan (CT scan with contrast may also be used to determine the presence of brain lesions if MRI is contraindicated) at screening to identify any untreated brain metastases (Exclusion criterion #3). Brain scan is not required with every subsequent disease assessment, regardless of history of prior brain metastases, and is performed if clinically indicated, according to local guidelines and practices.

If a participant is deriving clinical benefit and treatment beyond documented disease progression is approved, disease assessments continues as scheduled and clinical benefit after each disease assessment is reviewed.

### Safety Assessments

Any clinically significant abnormalities persisting at the end of the study/early withdrawal are followed until resolution or until a clinically stable condition is reached.

The study includes the following evaluations of safety and tolerability according to the time points provided in the Schedule of Activities Section.

### Physical Examinations

The screening physical examination includes, at a minimum, the participant's height, weight, and general appearance and an examination of the skin, ears, nose, throat, lungs, heart, abdomen, extremities, musculoskeletal system, lymphatic system, and nervous system. On Day 1 of each cycle, directed physical examinations of involved organs and other body systems, as indicated, are performed and participant body weight is obtained using a calibrated scale.

### Vital Signs

Vital sign measurements include the following assessments as indicated in the Schedule of Activities (Table 1):
Temperature
Heart rate
Respiratory rate
Oxygen saturation
Blood pressure

Blood pressure and heart rate measurements are assessed in a seated position with a completely automated device. Manual techniques are used only if an automated device is not available.

Blood pressure and heart rate measurements are preceded by at least 5 minutes of rest in a quiet setting without distractions (e.g., television, cell phones).

### Electrocardiograms

Triplicate electrocardiograms (ECGs), performed locally, are collected at screening to determine the eligibility. During the collection of ECGs, participants are in a quiet setting without distractions (e.g., television, cell phones). Participants rest in a supine position for at least 5 minutes before ECG collection and are refrain from talking or moving arms or legs. If blood sampling or vital sign measurement is scheduled for the same time point as ECG recording, the procedures are performed in the following order: ECG(s), vital signs, blood draw.

Three individual ECG tracings are obtained as closely as possible in succession, but approximately 2 minutes apart. The ECG, including ECG morphology, is reviewed for immediate management.

QTcF is calculated using the Fridericia's formula: QTcF=QT/(RR)\hat{ }0.33.

### ECOG Performance Status

Eastern Cooperative Oncology Group performance status score is evaluated during the screening phase to determine the eligibility.

### Clinical Safety Laboratory Assessments

Clinical laboratory assessments are performed locally. Clinical laboratory tests are performed as noted in Table 5.

TABLE 5
Clinical laboratory assessments

| Laboratory | | |
|---|---|---|
| Assessments | Parameters | |
| Hematology | Hemoglobin | Absolute neutrophil count |
| | Platelet count | White blood cell (WBCs) |
| | | absolute count with |
| | | differential |
| Clinical | Alkaline phosphatase | Total protein |
| Chemistry | Creatinine | Blood ureanitrogen |
| | Aspartate aminotransferase | Magnesium |
| | Alanine aminotransferase | Phosphorus |
| | Gamma-glutamyl transferase | Sodium |
| | Bilirubin (total, direct, indirect) | Potassium |
| | Lactic acid dehydrogenase | Calcium |
| | Albumin | |
| Coagulation | Prothrombin time (in seconds) | International |
| (screening | | normalized ratio |
| only) | Activated partial thromboplastin | |
| | time | |
| Urinalysis | Dipstick | Sediment (if dipstick |
| (screening | | result is abnormal) |
| only) | Specific gravity | Red blood cells |
| | pH | WBCs |
| | Glucose | Epithelial cells |
| | Protein | Crystals |
| | Blood | Casts |
| | Ketones | Bacteria |
| | Bilirubin | |
| | Urobilinogen | |
| | Nitrite | |
| | Leukocyte esterase | |

| | |
|---|---|
| Serology | Anti-human immunodeficiency virus (anti-HIV) antibody |
| | hepatitis B surface antigen (HBsAg), hepatitis B surface |
| | antibody (HBsAb), and hepatitis B core antibody (HBcAb) |
| | (Participants with a history of hepatitis B virus (HBV) are |
| | also required to have HBV DNA quantification.) |
| | Anti-hepatitis C virus (anti-HCV) antibody |
| | (Participants with a history of HCV are required to have |
| | HCV RNA quantification.) |

More frequent clinical laboratory tests may be performed as indicated by the overall clinical condition of the participant or abnormalities that warrant more frequent monitoring.

### Pharmacokinetics

Blood samples are used to evaluate the PK of amivantamab. Serum collected for PK may additionally be used to evaluate safety or efficacy aspects that address concerns arising during or after the study period.

### Evaluations

Blood samples are collected for measurement of serum amivantamab for PK analyses. The PK profile of amivantamab is based on serum concentration data obtained from the timepoints surrounding the first and fifth dose administrations collected from at least 10 participants in each cancer type in Phase 2a. Blood samples for sparse PK is also obtained following all other dose administrations from participants in Phase 2a and 2b, prior to the start of the infusion and following the end of the infusion, from all the participants. Analytical

### Procedures

Pharmacokinetics: Serum samples are analyzed to determine concentrations of amivantamab using a validated, specific, and sensitive enzyme-linked immunosorbent assay (ELISA) method.

In addition, serum PK samples may be stored for future analysis of other co-administered treatments.

### Pharmacokinetic Parameters and Evaluations

The primary PK endpoints include, but are not limited to maximum serum concentration (Cₘₐₓ), Tₘₐₓ, AUC(ₜ₁₋ₜ₂) (e.g., AUC_{Day1-8}), AUCₜₐᵤ, plasma/serum concentration immediately prior the next study treatment administration (C_{trough}), t_{1/2}, CL, steady state volume of distribution (Vss), and accumulation ratio. Population PK modeling may be performed to assess the potential effect of intrinsic factors and extrinsic on the PK of amivanatamab.

### Biomarkers

Collected tumor tissue samples are used to evaluate the tumor surface levels of EGFR and cMET protein expression by centrally performed IHC assay to determine the patient eligibility, although documentation of previously performed local IHC results may be submitted for the purposes of demonstrating eligibility for study conduct. All statistical and biomarker analysis, however, utilize the results of the centrally performed IHC results, which classify patients as 0, 1+, 2+ or 3+ based on the highest staining of either EGFR or cMet. Tumor tissue collected at screening may also be analyzed by tumor next-generation sequencing to evaluate molecular alterations and track response to treatment. Tumor samples collected post-treatment and post-progression may also be evaluated by IHC and next-generation sequencing to track response to amivantamab. Tissues may also be used to determine biomarkers relevant to GC/EC and/or analyzed to confirm ctDNA results.

Screening blood samples from all participants undergo ctDNA analysis to evaluate pre-treatment mutational status of EGFR, cMet, and other key oncogenes to characterize the tumor. Additional blood samples are collected during the study and may be evaluated for ctDNA to assess changes in the levels or types of genetic alterations observed over time and to monitor for the emergence of potential markers of resistance to amivantamab.

Blood samples are also collected at time points and may be analyzed for circulating factors relevant to disease biology (e.g., hepatocyte growth factor).

Blood samples are also collected from at least 10 participants in each cancer type at selected time points to analyze PD markers (e.g., soluble EGFR and cMet) in samples taken prior to and after exposure to amivantamab, to explore whether the complete soluble target saturation throughout the dosing was attained.

For the provision of biopsy tissue samples, formalin-fixed, paraffin-embedded (FFPE) tissue samples are requested and are evaluated for biomarkers (DNA, RNA, and/or protein) relevant to cancer.

### Immunogenicity Assessments

Serum samples are collected for immunogenicity assessments of amivantamab (anti-drug antibodies to amivantamab). The detection and characterization of antibodies to amivantamab is performed using a validated immunoassay method.

Serum samples are screened for antibodies binding to amivantamab and serum titer is determined from positive samples. Antibodies may be further characterized and/or evaluated for their ability to neutralize the activity of the study treatment. All samples collected for immune response analysis are also evaluated for amivantamab serum concentration to ensure appropriate interpretation of immunogenicity data. Other immunogenicity analyses may be performed to further characterize any immune responses generated.

### Statistical Considerations

A general description of the statistical methods to be used to analyze the efficacy and safety data is outlined below.

### Statistical Hypotheses

No hypothesis is planned to be tested in Phase 2a.

The statistical hypothesis in Phase 2b is that amivantamab monotherapy leads to objective response rate (ORR) higher than 15% (i.e., H₀≤15% vs Hₐ>15%) in patients with GC or EC, selected on the basis of expression of EGFR, cMET, or both. This threshold is based on historical studies for approved 3L regimens for GC (11.2%-13.6%) and reported efficacy of approved 2L regimens for EC (approximately 15%).

### Sample Size Determination

In Phase 2a, approximately 30 response evaluable participants with tumors expressing either EGFR, cMet, or both, as determined by central IHC, are enrolled in GC and EC cohorts. Twenty participants are enrolled for IHC 2+/3+ that provides approximately 90% probability to observe the posterior probability of (ORR>22.5%)≥40% (which is similar with ORR≥20%) assuming ORR is 30% for the subpopulation. By enrolling 10 participants with IHC 1+, the probability to observe the posterior probability (ORR>22.5%)≥40% is 80%. A maximum of 11 participants may be enrolled in each Phase 2a extension cohort. Enrollment halts if no response or stable disease of 6 weeks or more is observed among the first 6 participants for futility in each of the Phase 2a extension cohorts. If 2 or more responses are observed in each of Phase 2a extension cohorts, Additional participants may be enrolled for further characterization.

In Phase 2b cohorts, approximately 100 participants are enrolled in each of GC and EC expansion cohort. The eligible participants are decided based on the results in Phase 2a part. Assuming an overall ORR of 30% for amivantamab, 100 participants in Phase 2b part provides approximately 90% power to reject the null hypothesis, 15% ORR, using 2-side z test at alpha=0.05.

### Populations for Analysis Sets

For purposes of analysis, the following populations are defined:
(1) All Treated population: All participants who take at least 1 dose of study treatment;
(2) Response evaluable population: All participants who satisfy the following criteria:
   Receive at least 1 dose of study treatment;
   At least 2 post-baseline efficacy disease assessments, or discontinued treatment for any reason, or have disease progression/death prior to the second post-baseline disease assessment;
(3) Safety population: All participants who take at least 1 dose of study treatment;
(4) Pharmacokinetics population: All participants who receive at least 1 dose of study treatment and have at least 1 evaluable post-baseline measurement;
(5) Immunogenicity population: All participants who receive at least 1 dose of study treatment and have at least 1 evaluable post-baseline measurement.

### General Considerations

All continuous variables are summarized using number of participants (n), mean, standard deviation (SD), median, minimum, and maximum. Discrete variables are summarized with number and percent. The Kaplan-Meier product limit method is used to estimate the time-to-event variables including median survival time. Unless otherwise specified, the phases and arms are analyzed separately.

Analyses of ORR and disease control rate (DCR) is performed on the response evaluable population. The other efficacy analyses are performed on all treated population. The central IHC data are used for the statistical analysis purposes.

At the end of Phase 2a part, the result are reviewed and which subpopulations to be included in the Phase 2b part are determined.

### Primary Endpoint

Objective Response Rate. Primary Estimand:
Population: participants with GC or EC expressing EGFR and/or MET
Variable: overall response; CR or PR
Study drug: amivantamab monotherapy
Intercurrent event: subsequent anticancer therapy. The while-on-treatment policy: Response after this intercurrent event is not included.
Summary: ORR

The primary efficacy measure is ORR. Objective response rate is defined as the proportion of participants who achieve either CR or PR, determined by investigator assessment using RECIST Version 1.1. Confirmation of investigator-assessed ORR may be performed through IRC in the Phase 2b.

For Phase 2a part, there is no formal hypothesis testing. ORR is calculated for response evaluable population descriptively.

For Phase 2b part, a z test with normal approximation is used to compare the ORR with 15%. Multiplicity caused by subpopulation selection at the interim analysis (see Interim Analysis Section) is controlled by closed testing procedure and weighted statistics. The ORR and its 95% confidence interval (CI) are also calculated.

### Secondary Endpoints

Disease Control Rate: Disease control rate is defined as the percentage of participants achieving complete or partial response or stable disease for at least 6 weeks as defined by RECIST Version1.1. The DCR and its 95% CI with Clopper-Pearson method are also calculated.

Duration of Response: Duration of Response (DoR) is defined as the time from the date of first documented response (CR or PR) until the date of documented progression or death, whichever comes first. The end of response would coincide with the date of progression or death from any cause used for the PFS endpoint. If a participant does not progress following a response, then his/her duration of response uses the PFS censoring time. A Kaplan-Meier plot and median DoR with 95% confidence interval (calculated from the Kaplan-Meier estimate) are presented. Confirmation of investigator-assessed DoR may be performed through IRC in the Phase 2b.

Progression-free Survival: Progression-free survival is defined as the time from first dose until the date of objective disease progression or death (by any cause in the absence of progression), whichever comes first, based on investigator assessment using RECIST Version 1.1. Participants who have not progressed or have not died at the time of analysis are censored at the time of the latest date of assessment from their last evaluable RECIST Version 1.1 assessment. PFS is analyzed using the same methodology as for the analysis of DoR.

Overall Survival: Overall survival is defined as the time from the date of first dose until the date of death due to any cause. Any participant not known to have died at the time of analysis are censored based on the last recorded date on which the participant was known to be alive. OS is analyzed using the same methodology as for the analysis of DoR.

### Other Analyses

### Pharmacokinetic Analyses

The PK analyses use the PK population. Serum amivantamab concentrations are summarized for each cancer type and overall population in tables of mean, SD, median, and range over time, as appropriate. PK parameters are estimated for individuals and descriptive statistics are calculated for each cancer type and overall population.

Participants are excluded from the PK analysis if their data do not allow for accurate assessment of the PK (e.g., incomplete administration of the study treatment; missing information of dosing and sampling times; concentration data not sufficient for PK parameter calculation).

### Pharmacokinetic/Pharmacodynamic Analyses

The exposure-response relationship between amivantamab exposure and key efficacy and safety parameters may be explored if the data allow. In addition, the relationship may be characterized using an exposure-response model.

### Immunogenicity Analyses

The incidence of anti-amivantamab antibodies is summarized for immunogenicity population.

Serum samples are screened for antibodies binding to amivantamab and the number of confirmed positive samples are reported. Other analyses may be performed to further characterize the immunogenicity of amivantamab.

### Pharmacodynamic and Biomarker Analyses

The biomarker analyses use the biomarker population. Analyses are planned to explore PD and other biomarkers that may be indicative of the mechanisms of action of the drug or predictive of efficacy as well as the potential mechanisms of resistance to amivantamab.

The association of biomarker-positivity with clinical response or time-to-event endpoints is assessed using statistical methods appropriate for each endpoint (eg, analysis of variance, categorical, or survival models). Correlation of baseline biomarker expression levels with clinical response or relevant time to-event endpoints is performed to identify responsive (or resistant) subgroups.

Additional biomarkers (DNA, RNA, and/or protein) relevant to GC/EC may also be assessed in blood and tissue samples collected during the study to better understand the disease and mechanisms of response or resistance to amivantamab.

### Interim Analysis

In Phase 2b, an interim futility analysis is planned in each of GC and EC arm approximately 12 weeks after 50 participants receive the first infusion. The interim futility analyses is based on the best response rate for each subpopulation (for example, IHC 2+/3+ and IHC 1+) selected at the end of Phase 2a and prespecified before initiating Phase 2b). The enrollment of each subpopulation may be terminated for futility if the posterior probability (ORR>22.5%) is <40%.

### Results.

Participants in Phase 2a gastric cancer (GC) and esophageal cancer (EC) cohorts received intravenous (IV) infusion of weight-based dose of amivantamab in 28-day cycles. Participants with body weight less than (<) 80 kilograms (kg) received IV infusion of amivantamab 1,050 milligrams (mg) and participants with body weight greater than or equal to (≥) 80 kg received IV infusion of amivantamab 1,400 mg once weekly in Cycle 1 and then every 2 weeks in subsequent cycles (on Days 1 and 15 of each cycle). The enrollment status is shown in Table 6. The demographics and disease characteristics are shown in Table 7. The overall safety summary of treatment emergent adverse events (TEAEs) are shown in Table 8. The TEAEs related to grade 3 or higher are shown in Table 9.

TABLE 6
Enrollment Status

| | Gastric cancer | Esophageal cancer |
|---|---|---|
| All treated patients | 29 | 30 |
| IHC result | IHC ≥ 2: 20, | IHC ≥ 2: 27, |
| | IHC 1: 5, NA: 4 | IHC 1: 1, NA: 2 |
| Subjects ongoing | 0 | 2 (6.7%) |
| Discontinued study treatment | 29 (100%) | 28 (93.3%) |
| Reason for discontinuation | | |
| Progressive disease | 24 (82.8%) | 22 (73.3%) |
| Adverse event | 1 (3.4%) | 2 (6.7%) |
| Subject refused further | 3 (10.3%) | 2 (6.7%) |
| study treatment | | |
| Physician decision | 0 | 1 (3.3%) |
| Death | 1 (3.4%) | 0 |
| Other | 0 | 1 (3.3%) |

TABLE 7
Demographics and disease characteristics

| | Gastric cancer | Esophageal cancer |
|---|---|---|
| | N = 29 | N = 30 |
| Median age, years | 66.0 | 60.0 |
| Male/Female | 24 (82.8%)/5 (17.2%) | 24 (80.0%)/6 (20.0%) |
| Histology | | |
| Adenocarcinoma | 29 (100%) | 1 (3.3%) |
| Squamous cell carcinoma | 0 | 29 (96.7%) |
| Other | 0 | 0 |
| Location of disease | | |
| Lymph node | 26 (89.7%) | 26 (86.7%) |
| Liver | 16 (55.2%) | 11 (36.7%) |
| Brain | 0 | 0 |
| Bone | 1 (3.4%) | 2 (6.7%) |
| Peritoneum | 10 (34.5%) | 2 (6.7%) |
| Lung | 6 (20.7%) | 15 (50.0%) |
| Adrenal gland | 2 (6.9%) | 0 |
| Other | 3 (10.39%) | 5 (16.7%) |
| Median number of prior lines | 3.0 (2-6) | 3.0 (1-9) |
| (range) | | |
| Platinum-based chemotherapy | 29 (100%) | 30 (100%) |
| Fluoropyrimidine | 29 (100%) | 30 (100%) |
| Immune checkpoint inhibitor | 21 (72.4%) | 25 (83.3%) |
| HER2 targeted therapy | 7 (24.1%) | 1 (3.3%) |

TABLE 8
Overall safety summary of TEAEs

| | Gastric | Esophageal |
|---|---|---|
| All treated population (safety | 29 | 30 |
| analysis set) | | |
| Subjects with 1 or more: | | |
| AEs | 29 (100.0%) | 30 (100.0%) |
| Related AEs | 27 (93.1%) | 29 (96.7%) |
| SAEs | 9 (31.0%) | 7 (23.3%) |
| Related SAEs | 0 | 2 (6.7%) |
| AEs leading to death | 3 (10.3%) | 1 (3.3%) |
| Related AEs leading to death | 0 | 1 (3.3%) |
| AEs leading to discontinuation | 1 (3.4%) | 1 (3.3%) |
| of study agent | | |
| Related AEs leading to | 1 (3.4%) | 1 (3.3%) |
| discontinuation of study agent | | |
| AEs leading to study treatment | 15 (51.7%) | 18 (60.0%) |
| interruption | | |
| Related AEs leading to study | 15 (51.7%) | 15 (50.0%) |
| treatment interruption | | |
| AEs leading to dose reduction | 2 (6.9%) | 4 (13.3%) |
| Related AEs leading to dose | 2 (6.9%) | 4 (13.3%) |
| reduction | | |
| AEs leading to dose delay | 0 | 2 (6.7%) |
| Related AEs leading to dose delay | 0 | 1 (3.3%) |

TABLE 9
≥ Grade 3 Related TEAEs

| | Gastric | Esophageal |
|---|---|---|
| Subjects with 1 or more related ≥ Grade 3 | 2 (6.9%) | 4 (13.3%) |
| Related AEs: | | |

Preferred term

| | | |
|---|---|---|
| Hypoalbuminemia | 2 (6.9%) | 2 (6.7%) |
| Neutropenia | 0 | 1 (3.3%) |
| Interstitial lung disease | 0 | 1 (3.3%) |

The observed incidence of infusion related reactions (IRRs) was similar to other amivantamab monotherapy studies. The incidence of hypoalbuminemia in the gastric cohort was slightly higher than that of esophageal cohort:

$$Gatric ( n = 29 ) : All grade : 12 ( 41.4 \% ) , \geq Grade 3 : 3 ( 10.3 \% ) \\ Esophageal ( n = 30 ) : All grade : 10 ( 33.3 \% ) , \geq Grade 3 : 3 ( 10. \% )$$

Next, response to treatment was evaluated. The “All Treated” patient population were patients who received at least 1 dose of study treatment. The “Response Evaluable” patient population were patients who (1) received at least 1 dose of study treatment, (2) met all eligibility criteria for the study, and (3) had a baseline and at least 1 post-baseline efficacy disease assessments, or have disease progression/death due to disease progression prior to the first post-baseline disease assessment. Table 10 shows summary of objective response rate based on RECIST Version 1.1 criteria by pre-screening IHC Score (Central) in response evaluable population. Table 11 shows summary of objective response rate based on RECIST Version 1.1 criteria by pre-screening IHC score (Central) in all treated population.

TABLE 10
Summary of Objective Response Rate Based on RECIST Version 1.1 Criteria by Pre-screening
IHC Score (Central) - Investigator Judgement (Response evaluable population)

| | Gastric Cancer | | | | | | Esophageal Cancer | | | | | |
|---|---|---|---|---|---|---|---|---|---|---|---|---|
| | IHC Score | | | | | | IHC Score | | | | | |
| | 3+ | 2+ | 1+ | 0 | NA | Total | 3+ | 2+ | 1+ | 0 | NA | Total |
| Analysis set: | 9 | 11 | 4 | 0 | 0 | 24 | 17 | 10 | 1 | 0 | 10 | 28 |
| Response | | | | | | | | | | | | |
| Evaluable | | | | | | | | | | | | |
| Best overall | | | | | | | | | | | | |
| response | | | | | | | | | | | | |
| N | 9 | 11 | 4 | 0 | 0 | 24 | 17 | 10 | 1 | 0 | 0 | 28 |
| Complete | 0 | 0 | 0 | — | — | 0 | 0 | 0 | 0 | — | — | 0 |
| response (CR) | | | | | | | | | | | | |
| Partial | 1 | 0 | 0 | — | — | 1 | 2 | 1 | 0 | — | — | 3 |
| response (PR) | (11.1%) | | | | | (4.2%) | (11.8%) | (10.0%) | | | | (10.7%) |
| Stable disease | 3 | 1 | 1 | — | — | 5 | 9 | 6 | 1 | — | — | 16 |
| (SD)^{a} | (33.3%) | (9.1%) | (25.0%) | | | (20.8%) | (52.9%) | (60.0%) | (100.0%) | | | (57.1%) |
| Progressive | 5 | 9 | 2 | — | — | 16 | 6 | 3 | 0 | — | — | 9 |
| Disease (PD) | (55.6%) | (81.8%) | (50.0%) | | | (66.7%) | (35.3%) | (30.0%) | | | | (32.1%) |
| Not Evaluable | 0 | 1 | 1 | — | — | 2 | 0 | 0 | 0 | — | — | 0 |
| (NE) | | (9.1%) | (25.0%) | | | (8.3%) | | | | | | |
| Objective | 1 | 0 | 0 | — | — | 1 | 2 | 1 | 0 | — | — | 3 |
| response rate | (11.1%) | | | | | (4.2%) | (11.8%) | (10.0%) | | | | (10.7%) |
| (CR + PR) | | | | | | | | | | | | |
| 95% CI^{b} | (0.3%, | (0.0%, | (0.0%, | — | — | (0.1%, | (1.5%, | (0.3%, | (0.0%, | — | — | (2.3%, |
| | 48.2%) | 28.5%) | 60.2%) | | | 21.1%) | 36.4%) | 44.5%) | 97.5%) | | | 28.2%) |
| Posterior | 0.305 | 0.047 | 0.280 | — | — | 0.014 | 0.195 | 0.254 | 0.601 | — | — | 0.082 |
| probability | | | | | | | | | | | | |
| (ORR >22.5%)^{c} | | | | | | | | | | | | |
| Disease | 4 | 1 | 1 | — | — | 6 | 11 | 7 | 1 | — | — | 19 |
| control rate | (44.4%) | (9.1%) | (25.0%) | | | (25.0%) | (64.7%) | (70.0%) | (100.0%) | | | (67.9%) |
| (CR + PR + SD) | (13.7%, | (0.2%, | (0.6%, | | | | | | | | | |
| 95% CI^{b} | 78.8%) | 41.3%) | 80.6%) | — | — | (9.8%, | (38.3%, | (34.8%, | (2.5%, | — | — | (47.6%, |
| | | | | | | 46.7% | 85.8%) | 93.3) | 100.0%) | | | 84.1%) |

Key:
CI = confidence interval
^{a}For a response to qualify as stable disease, follow-up measurements must have met the stable disease criteria at least once at a minimum interval ≥6 weeks after the first dose of study agent.
^{b}The exact Clopper-Pearson 95% CI is used.
^{c}The Posterior probability (ORR >22.5%) is calculated based on beta distribution.
Note:
A response of PR or CR must be confirmed by repeat assessments ≥4 weeks from the initial observation.
Note:
IHC score is defined as the highest staining of either EGFR or cMet.

TABLE 11
Summary of Objective Response Rate Based on RECIST Version 1.1 Criteria by Pre-
screening IHC Score (Central) - Investigator Judgement; (All treated population)

| | Gastric Cancer | | | | | | Esophageal Cancer | | | | | |
|---|---|---|---|---|---|---|---|---|---|---|---|---|
| | IHC Score | | | | | | IHC Score | | | | | |
| | 3+ | 2+ | 1+ | 0 | NA | Total | 3+ | 2+ | 1+ | 0 | NA | Total |
| Analysis | 9 | 11 | 5 | 0 | 4 | 29 | 17 | 10 | 1 | 0 | 2 | 30 |
| set: All | | | | | | | | | | | | |
| Treated | | | | | | | | | | | | |
| Best overall | | | | | | | | | | | | |
| response | | | | | | | | | | | | |
| N | 9 | 11 | 5 | 0 | 4 | 29 | 17 | 10 | 1 | 0 | 2 | 30 |
| Complete | 0 | 0 | 0 | — | 0 | 0 | 0 | 0 | 0 | — | 0 | 0 |
| response (CR) | | | | | | | | | | | | |
| Partial | 1 | 0 | 0 | — | 0 | 1 | 2 | 1 | 0 | | 0 | 3 |
| response (PR) | (11.1%) | | | | | (3.4%) | (11.8%) | (10.0%) | | | | (10.0%) |
| Stable | 3 | 1 | 1 | — | 1 | 6 | 9 | 6 | 1 | — | 2 | 18 |
| disease (SD)^{a} | (33.3%) | (9.1%) | (20.0%) | | (25.0%) | (20.7%) | (52.9%) | (60.0%) | (100.0%) | | (100.0%) | (60.0%) |
| Progressive | 5 | 9 | 2 | — | 3 | 19 | 6 | 3 | 0 | — | 0 | 9 |
| Disease (PD) | (55.6%) | (81.8%) | (40.0%) | | (75.0%) | (65.5%) | (35.3%) | (30.0%) | | | | (30.0%) |
| Not | 0 | 1 | 2 | — | 0 | 3 | 0 | 0 | 0 | — | 0 | 0 |
| Evaluable (NE) | | (9.1%) | (40.0%) | | | (10.3%) | | | | | | |
| Objective | 1 | 0 | 0 | — | 0 | 1 | 2 | 1 | 0 | — | 0 | 3 |
| response rate | (11.1%) | | | | | (3.4%) | (11.8%) | (10.0%) | | | | (10.0%) |
| (CR + PR) | | | | | | | | | | | | |
| 95% CI^{b} | (0.3%, | (0.0%, | (0.0%, | — | (0.0%, | (0.1%, | (1.5%, | (0.3%, | (0.0%, | — | (0.0%, | (2.1%, |
| | 48.2%) | 28.5%) | 52.2%) | | 60.2%) | 17.8%) | 36.4%) | 44.5%) | 97.5%) | | 84.2%) | 26.5%) |
| Posterior | 0.305 | 0.047 | 0.217 | — | 0.280 | 0.005 | 0.195 | 0.254 | 0.601 | — | 0.465 | 0.059 |
| probability | | | | | | | | | | | | |
| (ORR >22.5%)^{c} | | | | | | | | | | | | |
| Disease | 4 | 1 | 1 | — | 1 | 7 | 11 | 7 | 1 | — | 2 | 21 |
| control rate | (44.4%) | (9.1%) | (20.0%) | | (25.0%) | (24.1%) | (64.7%) | (70.0%) | (100.0%) | | (100.0%) | (70.0%) |
| (CR + PR + SD) | | | | | | | | | | | | |
| 95% CI^{b} | (13.7%, | (0.2%, | (0.5%, | — | (0.6%, | (10.3%, | (38.3%, | (34.8%, | (2.5%, | — | (15.8%, | (50.6%, |
| | 78.8%) | 41.3%) | 71.6%) | | 80.6%) | 43.5%) | 85.8%) | 93.3%) | 100.0%) | | 100.0%) | 85.3%) |

Key:
CI = confidence interval
^{a}For a response to qualify as stable disease, follow-up measurements must have met the stable disease criteria at least once at a minimum interval ≥6 weeks after the first dose of study agent.
^{b}The exact Clopper-Pearson 95% CI is used.
^{c}The Posterior probability (ORR >22.5%) is calculated based on beta distribution.
Note:
A response of PR or CR must be confirmed by repeat assessments ≥4 weeks from the initial observation.
Note:
IHC score is defined as the highest staining of either EGFR or cMet.

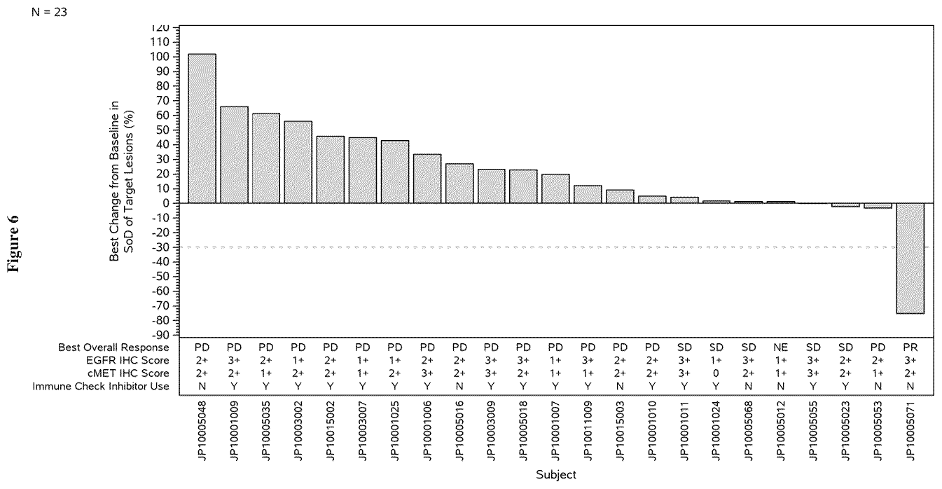

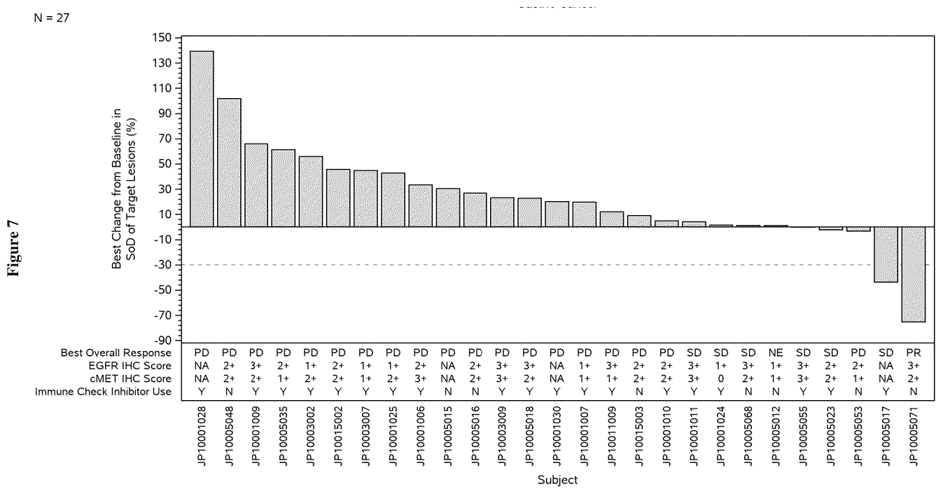

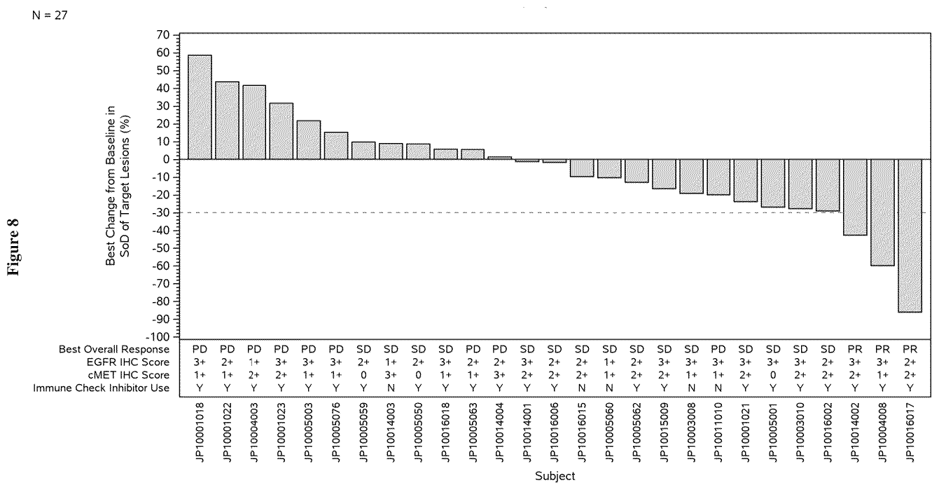

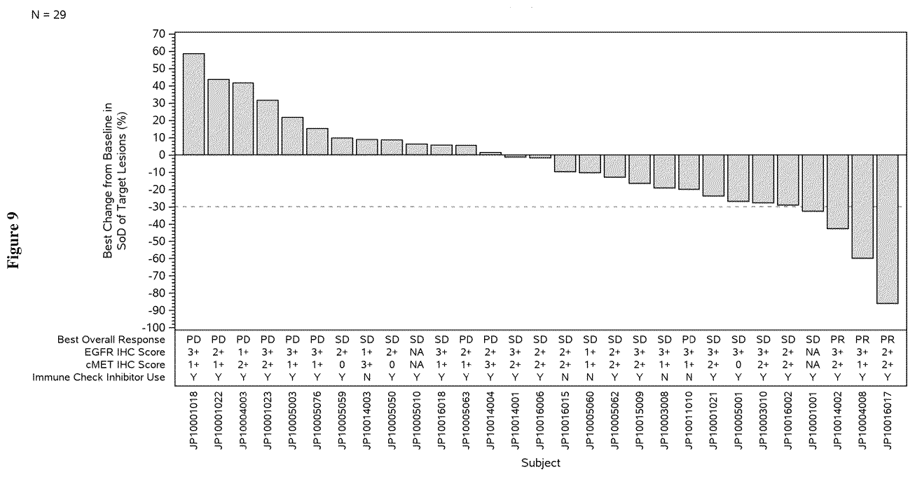

The overall response for gastric cancer patients in response evaluable population is shown in FIG. 6. The overall response for gastric cancer patients in all treated population is shown in FIG. 7. The overall response for esophageal cancer patients in response evaluable population is shown in FIG. 8. The overall response for esophageal cancer patients in all treated population is shown in FIG. 9.

In summary, safety profile in gastric/esophageal cancer cohorts is consistent with reported experience of other amivantamab monotherapy studies. Gastric cancer cohort had 1 partial response, 5 stable disease patients (disease control rate (DCR): 25.0%) among 24 response evaluable patients. Esophageal cancer cohort had 3 partial responses and 16 stable disease patients (DCR: 67.9%) among 28 response evaluable patients.

Higher dose of 1,750 mg amivantamab was evaluated in 3 patients with esophageal cancers. All 3 participants had body weight less than (<) 80 kilograms (kg) and received IV infusion of amivantamab 1,750 mg once weekly in Cycle 1, having the 1^{st} dose split over Day 1 and Day 2 as 350 mg and 1400 mg respectively, and then every 2 weeks in subsequent cycles (on Days 1 and 15 of each cycle). Table 12 shows demographic data and disease characteristics in patients treated with 1,750 mg amivantamab. Table 13 shows overall safety summary of TEAEs in patients treated with 1,750 mg amivantamab. No grade 3 or higher TEAEs were observed in the 1,750 mg amivantamab population.

TABLE 12
Demographics and disease characteristics

| | Esophageal cancer |
|---|---|
| | N = 3 |
| Median age, years | 60.0 |
| Male/Female | 3 (100%)/0 |
| Histology | |
| Adenocarcinoma | 0 |
| Squamous cell carcinoma | 2 (66.7%) |
| Other | 1 (33.3%) |
| Location of disease | |
| Lymph node | 3 (100%) |
| Liver | 1 (33.3%) |
| Brain | 1 (33.3%) |
| Bone | 0 |
| Peritoneum | 0 |
| Lung | 1 (33.3%) |
| Adrenal gland | 1 (33.3%) |
| Other | 1 (33.3%) |
| Median number of prior lines (range) | 5.0 (3-5) |
| Platinum-based chemotherapy | 3 (100%) |
| Fluoropyrimidine | 3 (100%) |
| Immune checkpoint inhibitor | 3 (100%) |
| HER2 targeted therapy | 0 |

TABLE 13
Overall safety summary of TEAEs.

| | 1750 mg amivantamab- |
|---|---|
| | trated patients |
| All treated population (safety analysis set) | 3 |
| Subjects with 1 or more: | |
| AEs | 3 (100.0%) |
| Related AEs | 3 (100.0%) |
| SAEs | 0 |
| Related SAEs | 0 |
| AEs leading to death | 0 |
| Related AEs leading to death | 0 |
| AEs leading to discontinuation of study | 0 |
| agent | |
| Related AEs leading to discontinuation | 0 |
| of study agent | |
| AEs leading to study treatment interruption | 2 (66.7%) |
| Related AEs leading to study treatment | 1 (33.3%) |
| interruption | |
| AEs leading to dose reduction | 0 |
| Related AEs leading to dose reduction | 0 |
| AEs leading to dose delay | 0 |
| Related AEs leading to dose delay | 0 |

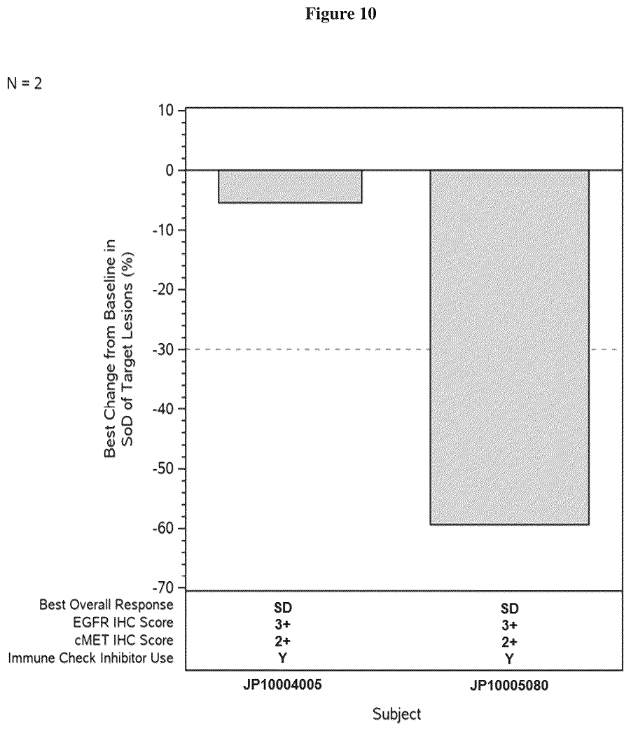

Efficacy was evaluated in 3 patients treated with 1750 mg amivantamab. One patient had stable disease, one had partial response, and one had progressing disease. FIG. 10 shows the best overall response in response evaluable patients.

### Example 2. Evaluation of Amivantamab in Esophageal Patient-Derived Xenograft Models (PDX) Models Expressing Wild-Type EGFR

To evaluate the efficacy of amivantamab in a series of esophageal PDX models (n=12, provided by CrownBio) carrying wild-type EGFR, tumor fragments from stock tumor bearing mice were harvested and inoculated into indicated experiment mice. Each mouse was inoculated subcutaneously in the right flank with indicated tumor fragments (2-3 mm in diameter) for tumor development. Detailed esophageal PDX inoculation information is listed in Table 14.

TABLE 14

| No. | Model ID | Cancer Type | Mouse Strain |
|---|---|---|---|
| 1 | ES0026 | Esophageal Carcinoma | BALB/c nude |
| 2 | ES0110 | Esophageal Carcinoma | BALB/c nude |
| 3 | ES0136 | Esophageal Carcinoma | BALB/c nude |
| 4 | ES0148 | Esophageal Carcinoma | BALB/c nude |
| 5 | ES0176 | Esophageal Carcinoma | BALB/c nude |
| 6 | ES0178 | Esophageal Carcinoma | BALB/c nude |
| 7 | ES11062 | Esophageal Carcinoma | NOD/SCID |
| 8 | ES11079 | Esophageal Carcinoma | BALB/c nude |
| 9 | ES11082 | Esophageal Carcinoma | NOD/SCID |
| 10 | ES2267 | Esophageal Carcinoma | BALB/c nude |
| 11 | ES3431 | Esophageal Carcinoma | BALB/c nude |
| 12 | ES3862 | Esophageal Carcinoma | BALB/c nude |

After establishment of palpable lesions, the tumor growth was measured twice weekly. Once the mean tumor volume reached approximate 150 mm³, animals were randomly allocated to relevant study groups with 8 mice per group. The randomization was performed according to the tumor size of each group, and the day of randomization was denoted as Day0. The treatments were started on the same day of randomization per study design in Table 15.

TABLE 15

| | | | Dosing | Dosing | Dosing |
|---|---|---|---|---|---|
| Group | N | Treatment | (mg/kg) | Route | Frequency |
| 1 | 8 | Isotype Control | 10 | i.p. | BIW |
| 2 | 8 | amivantamab | 10 | i.p. | BIW |

i.p.: intraperitoneal(ly); BIW: twice weekly (Day 0, 3 each week).

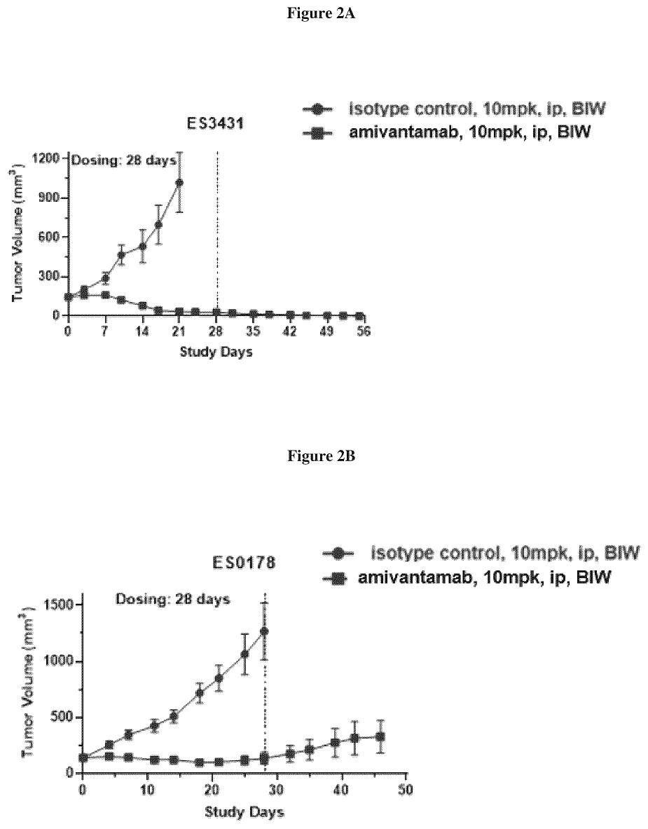

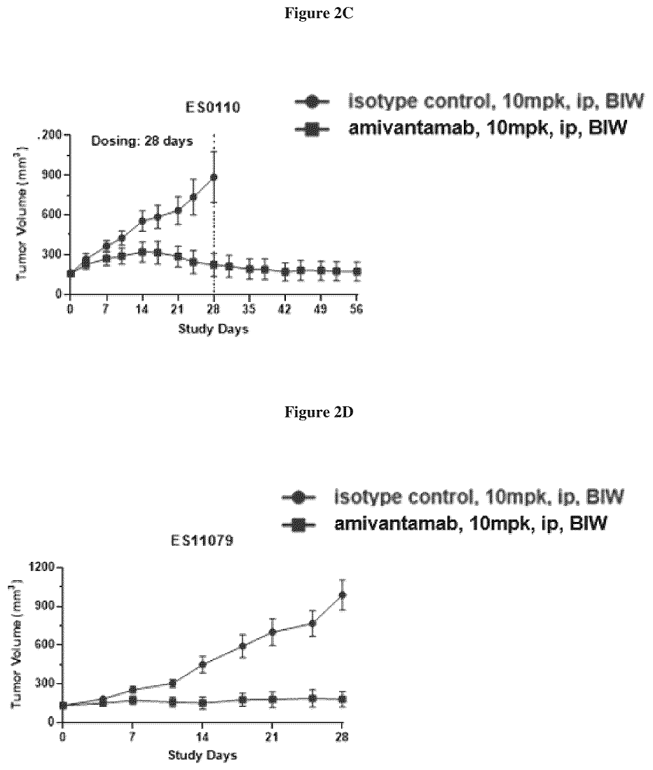

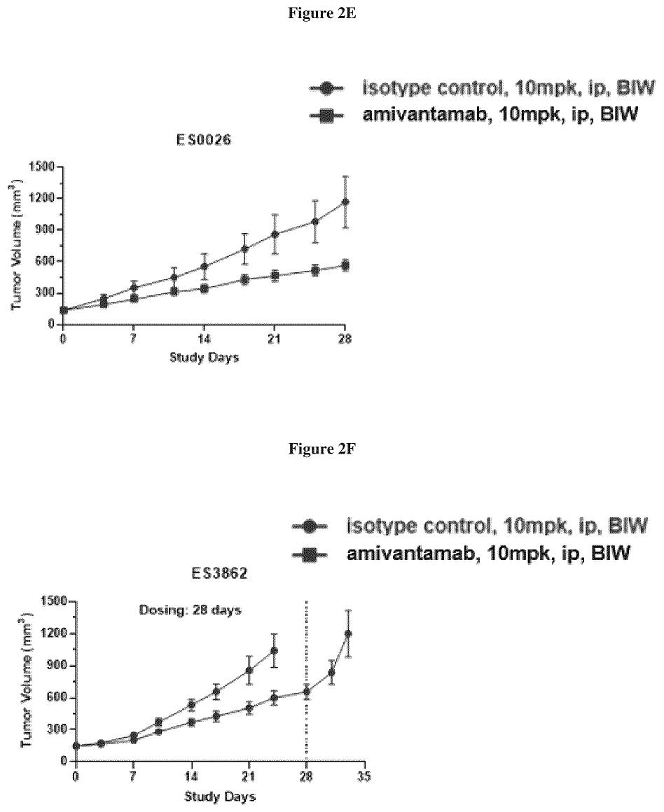

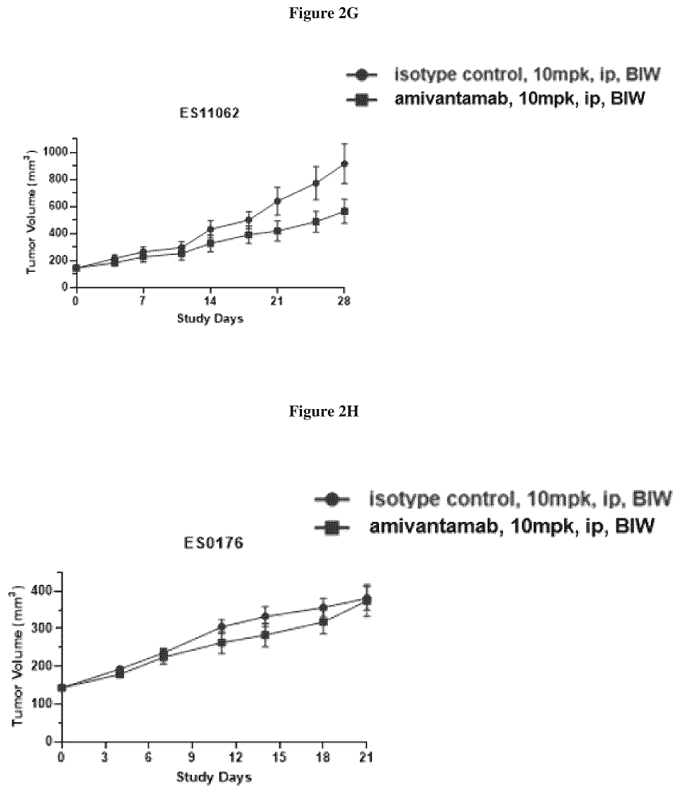

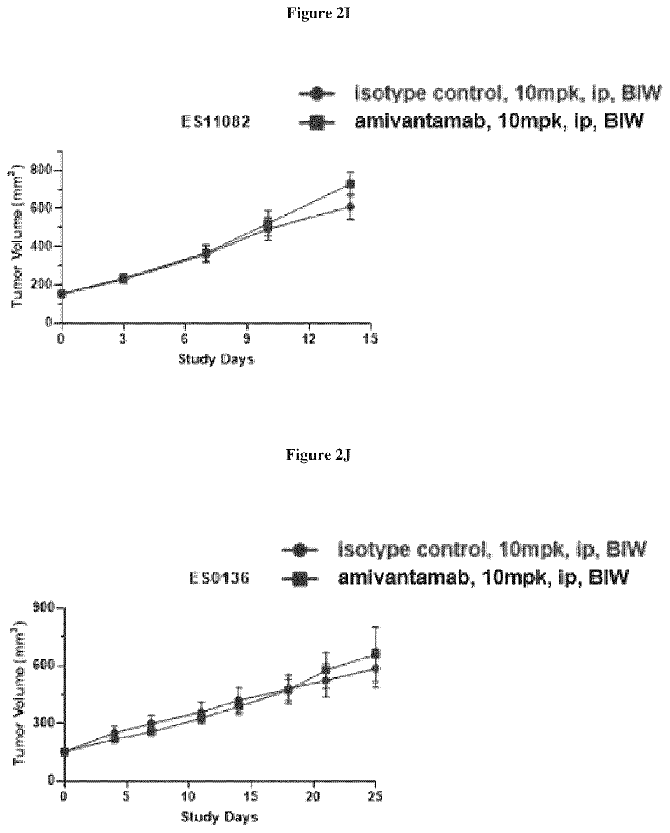

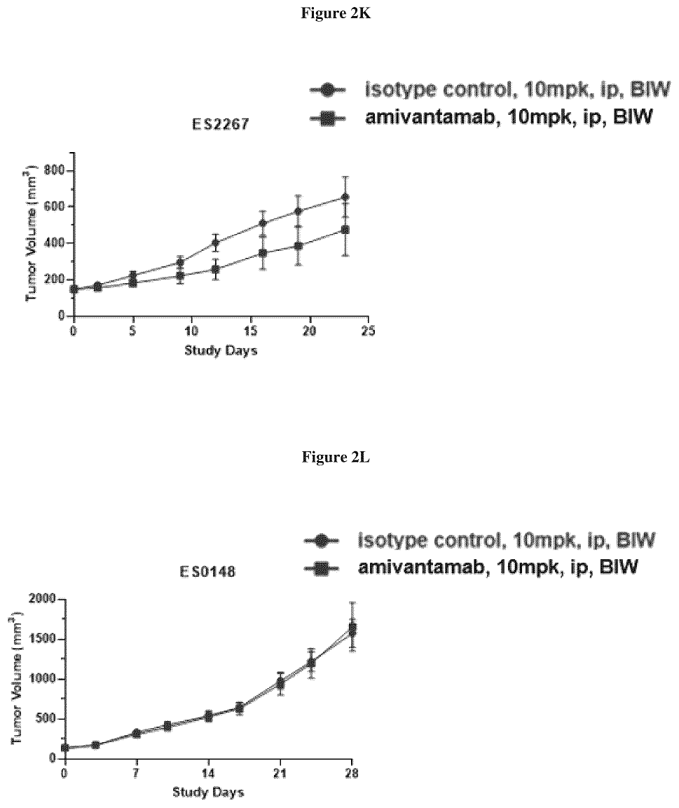

The study endpoints were to compare the tumor growth in each group at the end of treatments, and the subsequent tumor outgrowth after dosing stopped. The tumor size was measured twice weekly in two dimensions using a caliper, and the tumor volume was expressed in mm³ using the formula V=0.5×L×W², where V is tumor volume, L is tumor length (longest tumor dimension) and W is tumor width (longest tumor dimension perpendicular to L). The tumor growth curves (expressed as Mean±SEM) over time are shown in FIGS. 2A-2L.

Tumor growth inhibition (TG1%) was an indication of anti-tumor activity and calculated as TG1%=(1−(T−T0)/(C−C0))×100%, T and C were the mean tumor volume (TV) of treated and control groups, respectively on the day when mean TV of control group were terminated per study design. Tumor growth inhibition is summarized in Table 16.

TABLE 16

| | | Amivantamab |
|---|---|---|
| No. | Model ID | TGI%* |
| 1 | ES3431 | 112.68% |
| 2 | ES0178 | 100.40% |
| 3 | ES0110 | 90.76% |
| 4 | ES11079 | 94.47% |
| 5 | ES0026 | 58.57% |
| 6 | ES3862 | 49.36% |
| 7 | ES11062 | 45.51% |
| 8 | ES0176 | 2.97% |
| 9 | ES11082 | −26.05% |
| 10 | ES0136 | −17.16% |
| 11 | ES2267 | 35.48% |
| 12 | ES0148 | −5.59% |

*TGI% calculated as below:
$TGI \% = [ 1 - \frac{( T i - T 0 )}{( C i - C 0 )} ] \times 100 \%$ C (i or 0): Mean tumor volume of Control group on indicated Study Day.
T (i or 0): Mean tumor volume of Treatment group on indicated Study Day.

As shown in FIGS. 2A-2L and Table 16, 7 out of 12 esophageal PDX tumors responded to amivantamab mono-treatment. Among these, amivantamab induced regression of tumors in ES3431 (TG1%112.7%) and ES0178 (TG1%100.4%), strongly inhibited tumor growth in ES0110 (TG1%90.8%) and ES11079 (TG1%94.5%), and moderate response in ES0026, ES3862 and ES11062 (TG1%58.6%, 49.4%, and 45.5% respectively).

### Example 3. Evaluation of Amivantamab Single Agent or in Combination with Chemo-Drugs or Capmatinib in the Treatment of Gastric PDX Tumors

To evaluate the anti-tumor activity of amivantamab, a panel of gastric PDX models were selected for treatment with amivantamab alone, or in combination with c-Met inhibitor capmatinib (Selleck), as well as gastric chemo-regimens, such as 5-FU (Shanghai Xudong Haipu Pharm) plus cisplatin (Qilu Pharm), or paxlitaxel (Beijing Union Pharm).

Tumor fragments from stock tumor bearing mice were harvested and inoculated into BALB/c nude mice. Each mouse was inoculated subcutaneously in the right flank with indicated tumor fragments (2-3 mm in diameter) for tumor development. Detailed gastric PDX information is listed in Table 17.

TABLE 17

| No. | Model ID | Cancer Type | Mouse Strain |
|---|---|---|---|
| 1 | GA0046 | Gastric Carcinoma | BALB/c nude |
| 2 | GA0075 | Gastric Carcinoma | BALB/c nude |
| 3 | GA0152 | Gastric Carcinoma | BALB/c nude |
| 4 | GA2254 | Gastric Carcinoma | BALB/c nude |
| 5 | GA3121 | Gastric Carcinoma | BALB/c nude |
| 6 | GA3236 | Gastric Carcinoma | BALB/c nude |

After establishment of palpable lesions, the tumor growth was measured twice weekly. Once the mean tumor volume reached approximate 150 mm³, animals were randomly allocated to relevant study groups with 8 mice per group. The randomization was performed according to the tumor size of each group, and the day of randomization was denoted as Day0. The treatments were started on the same day of randomization according to the dosing regimen in Table 18 where appropriate.

TABLE 18

| | | | Dosing | Dosing | Dosing |
|---|---|---|---|---|---|
| Group* | N | Treatment | (mg/kg) | Route | Frequency |
| 1 | 8 | Isotype Control | 10 | i.p. | BIW |
| 2 | 8 | amivantamab | 10 | i.p. | BIW |
| 3 | 8 | 5-FU | 10 | i.p. | QD, 5 |
| | | | | | days/week* |
| | | cisplatin | 4 | i.p. | QW |
| 4 | 8 | 5-FU | 10 | i.p. | QD, 5 |
| | | | | | days/week |
| | | cisplatin | 4 | i.p. | QW |
| | | amivantamab | 10 | i.p. | BIW |
| 5 | 8 | paclitaxel | 10/15 | i.v. | QW |
| 6 | 8 | paclitaxel | 10/15 | i.v. | QW |
| | | amivantamab | 10 | i.p. | BIW |
| 7 | 8 | capmatinib | 10 | p.o. | BID |
| 8 | 8 | amivantamab | 10 | i.p. | BIW |
| | | capmatinib | 10 | p.o. | BID |

i.p.: intraperitoneal(ly); i.v.: intravenous(ly); p.o.: oral administration.
QW: once weekly; BIW: twice weekly (Day 0, 3 per week); BID: twice daily with 12 hours interval; QD, 5 days/week: once daily from Day 0-5 per week.
*GA0046 was treated with group 1, 2, 7, 8; GA0075 and GA0152 were treated with group 1-6 (15 mg/kg paclitaxel); GA3121 was treated with group 1, 2, 5, 6 (10 mg/kg paclitaxel); GA2254 and GA3236 were treated with group 1-2.

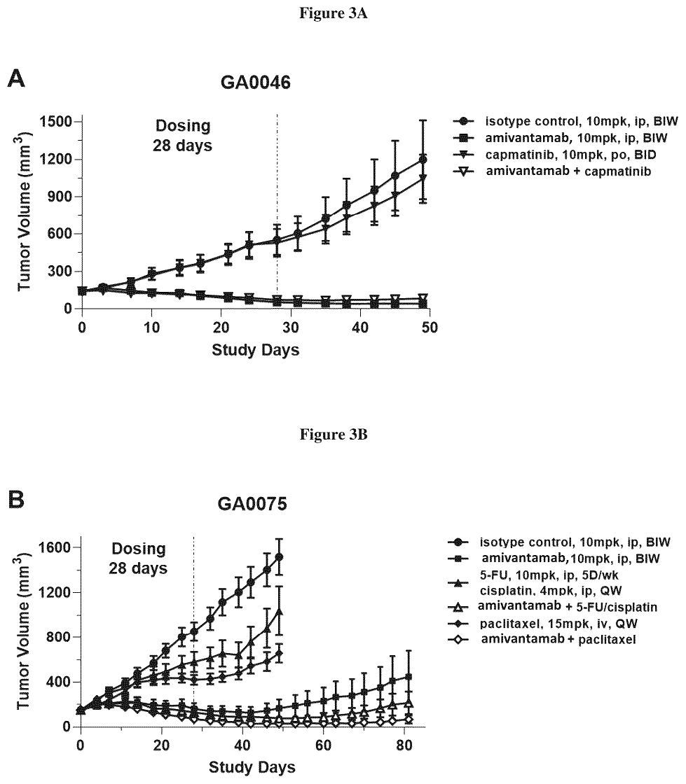

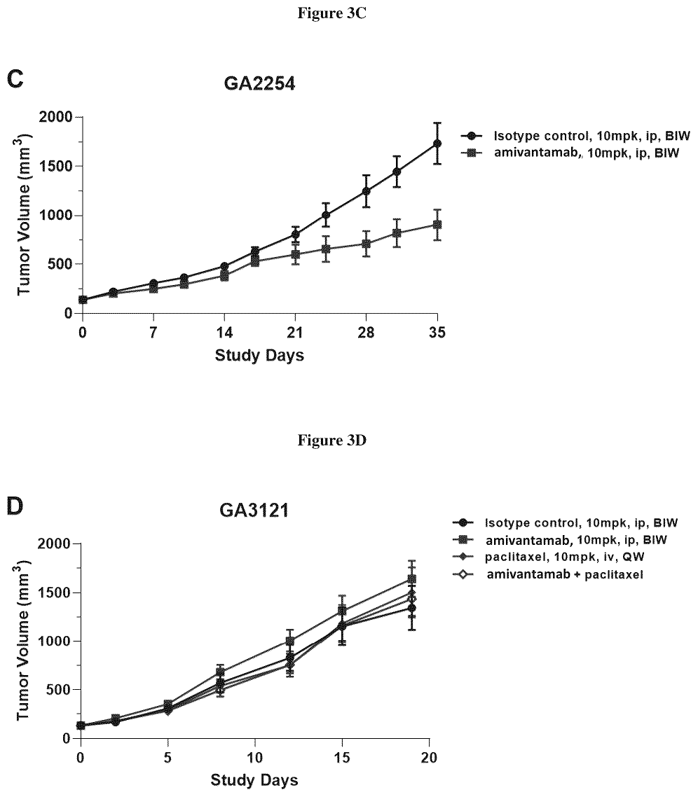

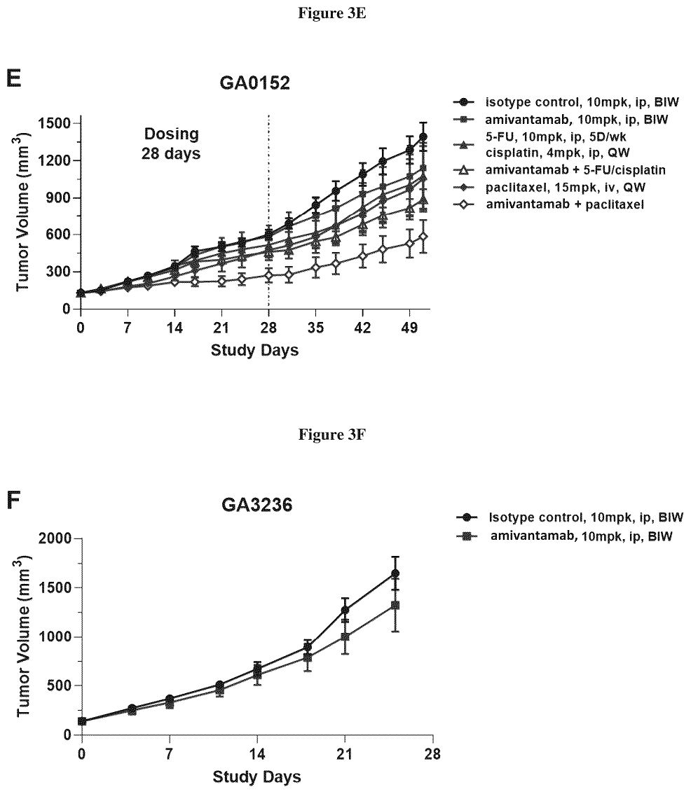

The study endpoints were to compare the tumor growth in each group at the end of treatments, and the subsequent tumor outgrowth after dosing stopped. The tumor size was measured twice weekly in two dimensions using a caliper, and the tumor volume was expressed in mm3 using the formula V=0.5×L×W², where V was tumor volume, L was tumor length (longest tumor dimension) and W was tumor width (longest tumor dimension perpendicular to L). Tumor growth curves (expressed as Mean±SEM) over time are shown in FIGS. 3A-3F.

Tumor growth inhibition (TGI %) was an indication of anti-tumor activities, and calculated as TGI %=(1−(T−T0)/(C−C0))×100%, T and C were the mean tumor volume (TV) of treated and control groups, respectively on the day when mean TV of control group were terminated per study design. Tumor growth inhibition are summarized in Table 19.

TABLE 19

| No. | Model ID | Treatment | TGI% * |
|---|---|---|---|
| 1 | GA0046 | Isotype Control | — |
| | | amivantamab | 121.31% |
| | | capmatinib | 5.93% |
| | | amivantamab + capmatinib | 116.86% |
| 2 | GA0075 | Isotype Control | — |
| | | amivantamab | 101.17% |
| | | 5-FU/cisplatin | 43.35% |
| | | amivantamab + 5-FU/cisplatin | 104.92% |
| | | paclitaxel | 66.09% |
| | | amivantamab + paclitaxel | 111.54% |
| 3 | GA2254 | Isotype Control | — |
| | | amivantamab | 52.17% |
| 4 | GA3121 | Isotype Control | — |
| | | amivantamab | −24.54% |
| | | paclitaxel | −12.99% |
| | | amivantamab + paclitaxel | −7.50% |
| 5 | GA0152 | Isotype Control | — |
| | | amivantamab | 3.84% |
| | | 5-FU/cisplatin | 18.35% |
| | | amivantamab + 5-FU/cisplatin | 30.25% |
| | | paclitaxel | 27.29% |
| | | amivantamab + paclitaxel | 70.13% |
| 6 | GA3236 | Isotype Control | — |
| | | amivantamab | 21.44% |

*TGI% calculated as below:
$TGI \% = [ 1 - \frac{( T i - T 0 )}{( C i - C 0 )} ] \times 100 \%$ C (i or 0): Mean tumor volume of Control group on indicated Study Day.
T (i or 0): Mean tumor volume of Treatment group on indicated Study Day.

As shown in FIGS. 3A-3F and Table 19, amivantamab mono-treatment (10 mg/kg BIW) inhibited tumor growth in 3 out of 6 PDX models with strong anti-tumor activity observed in GA0046 (TGI %121.31%) and GA0075 (101.17%).

In GA0046 (FIG. 3A), full tumor regression was observed with amivantamab single agent. In comparison, a c-Met inhibitor capmatinib (10 mg/kg BID) showed no effect on tumor growth (TG1%: 5.93% on Day28).

Standard of care chemo regimens were also evaluated in this study. As shown in FIG. 3B, 5-FU (10 mg/kg QD 5 days/week) plus cisplatin (4 mg/kg QW), or paclitaxel (10/15 mg/kg, QW) showed moderate activity, 43.35% and 66.09% on Day28 in GA0075. Strikingly, amivantamab combination elicited strong and long-lasting tumor growth inhibition in the same model (104.92% and 111.54% respectively). Similar synergistic effect between amivantamab and paxlitaxel was observed in another model GA0152, TGI % was improved from 27.29% to 70.13% (FIG. 3E).

### Example 4. Relationship Between Receptor Expression and Amivantamab In Vivo Efficacy in Esophageal and Gastric PDX Tumors

To further study anti-tumor activity of amivantamab versus the receptor level, immunohistochemistry assay was performed to examine membrane expression of both EGFR and c-Met. Paraffin-embedded tissues were sectioned into 4 μm slides and placed in an automatic staining system (Leica or Ventana). After a series of pre-set procedures (Dewax, peroxide block, primary antibody incubation, secondary antibody incubation, DAB reaction), stained slides (either with EGFR antibodies, clone 5B7 from Ventana or SP84 from Abcam, or with c-Met antibodies, clone SP44 from Ventana or Abcam) were further scanned with an image system (NanoZoomer) into high-resolution pictures for pathologist review and scoring.

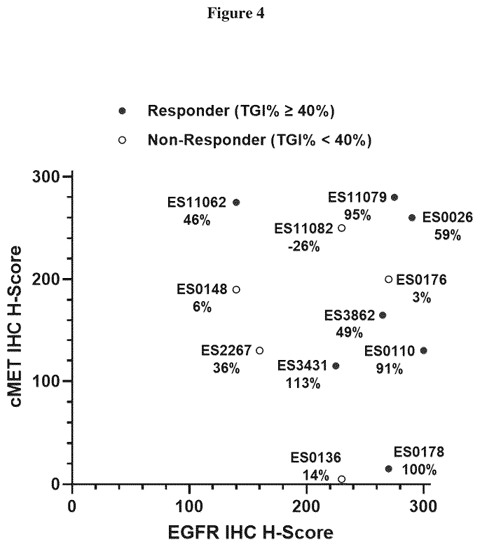

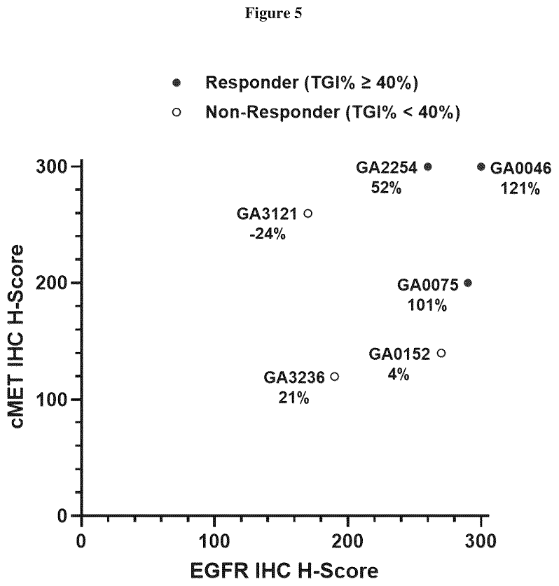

The intensity of EGFR or c-Met membrane staining from each slide was scored at four levels: 0, 1+, 2+, and 3+. Further H-score was calculated based on percentage of cells at different intensity levels with below equation. Relative EGFR and c-Met H-scores in tested esophageal and gastric PDX models, together with anti-tumor activities of amivantamab, were listed in Table 20, FIG. 4, and Table 21, FIG. 5, respectively. In the esophageal tumors, high EGFR levels appear to be associated with the tumor growth inhibition (TGI %), where in gastric tumors, high levels of EGFR and/or cMet appear to be associated with amivantamab TGI %.

$$H - Score ( 0 - 300 ) = ( \% @ 0 ) \times 0 + ( \% @ 1 ) \times 1 + ( \% @ 2 ) \times 2 + ( \% @ 3 ) \times 3 \\ \% @ 0 : percentage of cells with intensity 0 ( negative staining ) . \\ \% @ 1 : percentage of cells with intensity 1 + ( weak or incomplete membrane staining ) . \\ \% @ 2 : percentage of cells with intensity 2 + ( weak - to - moderate complete membrane staining ) . \\ \% @ 3 : percentage of cells with intensity 3 + ( strong complete membrane staining ) .$$

TABLE 20

| | | EGFR | c-Met | Amivantamab |
|---|---|---|---|---|
| No. | Model ID | H-score | H-score | TGI % |
| 1 | ES3431 | 225 | 115 | 112.68% |
| 2 | ES0178 | 270 | 15 | 100.40% |
| 3 | ES0110 | 300 | 130 | 90.76% |
| 4 | ES11079 | 275 | 280 | 94.47% |
| 5 | ES0026 | 290 | 260 | 58.57% |
| 6 | ES3862 | 265 | 165 | 49.36% |
| 7 | ES11062 | 140 | 275 | 45.51% |
| 8 | ES0176 | 270 | 200 | 2.97% |
| 9 | ES11082 | 230 | 250 | −26.05% |
| 10 | ES0136 | 230 | 5 | −17.16% |
| 11 | ES2267 | 160 | 130 | 35.48% |
| 12 | ES0148 | 140 | 190 | −5.59% |

TABLE 21

| | | EGFR | c-Met | Amivantamab |
|---|---|---|---|---|
| No. | Model ID | H-score | H-score | TGI % |
| 1 | GA0046 | 300 | 300 | 121.31% |
| 2 | GA0075 | 290 | 200 | 101.17% |
| 3 | GA2254 | 260 | 300 | 52.17% |
| 4 | GA3121 | 170 | 260 | −24.54% |
| 5 | GA0152 | 270 | 140 | 3.84% |
| 6 | GA3236 | 190 | 120 | 21.44% |

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A method of treating gastric cancer in a subject in need thereof, comprising administering to the subject a therapeutically effective amount of a bispecific anti-epidermal growth factor receptor (EGFR)/hepatocyte growth factor receptor (c-Met) antibody.

2. The method of claim 1, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

3. The method of claim 2, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

4. The method of claim 2, wherein the bispecific anti-EGFR/c-Met antibody is an IgG1 isotype.

5. The method of claim 1, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

6. The method of claim 1, wherein the bispecific anti-EGFR/c-Met antibody comprises a biantennary glycan structure with a fucose content of about between 1% to about 15%.

7. The method of claim 1, wherein the bispecific anti-EGFR/c-Met antibody is administered intravenously or subcutaneously to the subject.

8. The method of claim 7, wherein the bispecific anti-EGFR/c-Met antibody is administered at a dose of between about 350 mg to about 3400 mg.

9. The method of claim 8, wherein the bispecific anti-EGFR/c-Met antibody is administered at a dose of about 350 mg, 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2100 mg, 2200 mg, 2240 mg, 2300 mg, 2400 mg, 2500 mg, 2600 mg, 2700 mg, 2800 mg, 2900 mg, 3000 mg, 3100 mg, 3200 mg, 3300 mg, 3360 mg, or 3400 mg.

10. The method of claim 9, wherein the bispecific anti-EGFR/c-Met antibody is administered at a dose of 1050 mg.

11. The method of claim 9, wherein the bispecific anti-EGFR/c-Met antibody is administered at a dose of 1400 mg.

12. The method of claim 9, wherein the bispecific anti-EGFR/c-Met antibody is administered at a dose of 1600 mg.

13. The method of claim 9, wherein the bispecific anti-EGFR/c-Met antibody is administered at a dose of 1750 mg.

14. The method of claim 9, wherein the bispecific anti-EGFR/c-Met antibody is administered at a dose of 2100 mg.

15. The method of claim 9, wherein the bispecific anti-EGFR/c-Met antibody is administered at a dose of 2240 mg.

16. The method of claim 9, wherein the bispecific anti-EGFR/c-Met antibody is administered at a dose of 2400 mg.

17. The method of claim 9, wherein the bispecific anti-EGFR/c-Met antibody is administered at a dose of 3360 mg.

18. The method of claim 9, wherein the bispecific anti-EGFR/c-Met antibody is administered subcutaneously or intradermally to the subject.

19. The method of claim 9, wherein the bispecific anti-EGFR/c-Met antibody is administered intravenously to the subject.

20. The method of claim 1, wherein the bispecific anti-EGFR/c-Met antibody is administered twice a week, once a week, once in two weeks, once in three weeks or once in four weeks.

21. The method of claim 20, wherein the bispecific anti-EGFR/c-Met antibody is administered once a week for four weeks and once in two weeks thereafter.

22. The method of claim 21, wherein the first dose of the bispecific anti-EGFR/c-Met antibody is administered over two days.

23. The method of claim 1, wherein one or more cells of the gastric cancer express EGFR and/or cMet.

24. The method of claim 1, wherein the subject has received a prior treatment.

25. The method of claim 24, wherein the prior treatment comprises a chemotherapy, a targeted therapy, an immunotherapy, surgery, radiation therapy, chemoradiation therapy, or a combination thereof.

26. The method of claim 25, wherein the chemotherapy comprises a fluoropyrimidine-based chemotherapy, a platinum-based chemotherapy, paclitaxel, irinotecan, or a combination thereof.

27. The method of claim 26, wherein the fluoropyrimidine is 5-fluorouracil or capecitabine.

28. The method of claim 26, wherein the platinum-based chemotherapy is cisplatin, oxaliplatin, carboplatin, or nedaplatin.

29. The method of claim 25, wherein the targeted therapy comprises an anti-HER2 therapy or anti-VEGF/VEGFR therapy.

30. The method of claim 29, wherein the anti-HER2 therapy comprises trastuzumab.

31. The method of claim 30, wherein the anti-VEGF/VEGFR therapy comprises bevacizumab or ramucirumab.

32. The method of claim 1, wherein the subject is treatment naïve.

33. The method of claim 1, wherein the method further comprises administering at least one additional therapeutic to the subject.

34. The method of claim 33, wherein the additional therapeutic comprises a glucocorticosteroid, antihistamine, antipyretic, H₂-antagonist, antiemetic, opiate, or any combination thereof.

35. The method of claim 1, wherein the gastric cancer is an advanced or metastatic cancer.

36. The method of claim 1, wherein the subject is human.

37. A method of treating esophageal cancer in a subject in need thereof, comprising administering to the subject a therapeutically effective amount of a bispecific anti-epidermal growth factor receptor (EGFR)/hepatocyte growth factor receptor (c-Met) antibody.

38. The method of claim 37, wherein the bispecific anti-EGFR/c-Met antibody comprises a first domain that specifically binds EGFR and a second domain that specifically binds c-Met, wherein the first domain comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6, and wherein the second domain that binds c-Met comprises the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11 and the LCDR3 of SEQ ID NO: 12.

39. The method of claim 38, wherein the first domain that specifically binds EGFR comprises a heavy chain variable region (VH) of SEQ ID NO: 13 and a light chain variable region (VL) of SEQ ID NO: 14, and the second domain that specifically binds c-Met comprises the VH of SEQ ID NO: 15 and the VL of SEQ ID NO: 16.

40. The method of claim 38, wherein the bispecific anti-EGFR/c-Met antibody is an IgG1 isotype.

41. The method of claim 37, wherein the bispecific anti-EGFR/c-Met antibody comprises a first heavy chain (HC1) of SEQ ID NO: 17, a first light chain (LC1) of SEQ ID NO: 18, a second heavy chain (HC2) of SEQ ID NO: 19 and a second light chain (LC2) of SEQ ID NO: 20.

42. The method of claim 37, wherein the bispecific anti-EGFR/c-Met antibody comprises a biantennary glycan structure with a fucose content of about between 1% to about 15%.

43. The method of claim 37, wherein the bispecific anti-EGFR/c-Met antibody is administered intravenously or subcutaneously to the subject.

44. The method of claim 43, wherein the bispecific anti-EGFR/c-Met antibody is administered at a dose of between about 350 mg to about 3400 mg.

45. The method of claim 44, wherein the bispecific anti-EGFR/c-Met antibody is administered at a dose of about 350 mg, 700 mg, about 750 mg, about 800 mg, about 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2100 mg, 2200 mg, 2240 mg, 2300 mg, 2400 mg, 2500 mg, 2600 mg, 2700 mg, 2800 mg, 2900 mg, 3000 mg, 3100 mg, 3200 mg, 3300 mg, 3360 mg, or 3400 mg.

46. The method of claim 45, wherein the bispecific anti-EGFR/c-Met antibody is administered at a dose of 1050 mg.

47. The method of claim 45, wherein the bispecific anti-EGFR/c-Met antibody is administered at a dose of 1400 mg.

48. The method of claim 45, wherein the bispecific anti-EGFR/c-Met antibody is administered at a dose of 1600 mg.

49. The method of claim 45, wherein the bispecific anti-EGFR/c-Met antibody is administered at a dose of 1750 mg.

50. The method of claim 455, wherein the bispecific anti-EGFR/c-Met antibody is administered at a dose of 2100 mg.

51. The method of claim 455, wherein the bispecific anti-EGFR/c-Met antibody is administered at a dose of 2240 mg.

52. The method of claim 455, wherein the bispecific anti-EGFR/c-Met antibody is administered at a dose of 2400 mg.

53. The method of claim 455, wherein the bispecific anti-EGFR/c-Met antibody is administered at a dose of 3360 mg.

54. The method of claim 37, wherein the bispecific anti-EGFR/c-Met antibody is administered subcutaneously or intradermally to the subject.

55. The method of claim 46, wherein the bispecific anti-EGFR/c-Met antibody is administered intravenously to the subject.

56. The method of claim 37, wherein the bispecific anti-EGFR/c-Met antibody is administered twice a week, once a week, once in two weeks, once in three weeks or once in four weeks.

57. The method of claim 56, wherein the bispecific anti-EGFR/c-Met antibody is administered once a week for four weeks and once in two weeks thereafter.

58. The method of claim 57, wherein the first dose of the bispecific anti-EGFR/c-Met antibody is administered over two days.

59. The method of claim 37, wherein one or more cells of the esophageal cancer express EGFR and/or cMet.

60. The method of claim 37, wherein the subject has received a prior treatment.

61. The method of claim 60, wherein the prior treatment comprises a chemotherapy, a targeted therapy, an immunotherapy, surgery, radiation therapy, chemoradiation therapy, or a combination thereof.

62. The method of claim 61, wherein the chemotherapy comprises a fluoropyrimidine-based chemotherapy, a platinum-based chemotherapy, paclitaxel, irinotecan, or a combination thereof.

63. The method of claim 62, wherein the fluoropyrimidine is 5-fluorouracil or capecitabine.

64. The method of claim 62, wherein the platinum-based chemotherapy is cisplatin, oxaliplatin, carboplatin, or nedaplatin.

65. The method of claim 61, wherein the targeted therapy comprises an anti-HER2 therapy or anti-VEGF/VEGFR therapy.

66. The method of claim 65, wherein the anti-HER2 therapy comprises trastuzumab.

67. The method of claim 65, wherein the anti-VEGF/VEGFR therapy comprises bevacizumab or ramucirumab.

68. The method of claim 37, wherein the subject is treatment naïve.

69. The method of claim 37, wherein the method further comprises administering at least one additional therapeutic to the subject.

70. The method of claim 69, wherein the additional therapeutic is a glucocorticosteroid, antihistamine, antipyretic, H₂-antagonist, antiemetic, opiate, or any combination thereof.

71. The method of claim 37, wherein the esophageal cancer is an advanced or metastatic cancer.

72. The method of claim 37, wherein the subject is human.


SEQUENCE LISTING (20 sequences)

SEQ ID NO: 1 - protein, 5 aa, Homo sapiens
TYGMH

SEQ ID NO: 2 - protein, 17 aa, Homo sapiens
VIWDDGSYKYYGDSVKG

SEQ ID NO: 3 - protein, 16 aa, Homo sapiens
DGITMVRGVMKDYFDY

SEQ ID NO: 4 - protein, 11 aa, Homo sapiens
RASQDISSALV

SEQ ID NO: 5 - protein, 7 aa, Homo sapiens
DASSLES

SEQ ID NO: 6 - protein, 9 aa, Homo sapiens
QQFNSYPLT

SEQ ID NO: 7 - protein, 5 aa, Homo sapiens
SYGIS

SEQ ID NO: 8 - protein, 17 aa, Homo sapiens
WISAYNGYTNYAQKLQG

SEQ ID NO: 9 - protein, 10 aa, Homo sapiens
DLRGTNYFDY

SEQ ID NO: 10 - protein, 11 aa, Homo sapiens
RASQGISNWLA

SEQ ID NO: 11 - protein, 7 aa, Homo sapiens
AASSLLS

SEQ ID NO: 12 - protein, 9 aa, Homo sapiens
QQANSFPIT

SEQ ID NO: 13 - protein, 125 aa, Homo sapiens [sequence omitted]

SEQ ID NO: 14 - protein, 107 aa, Homo sapiens [sequence omitted]

SEQ ID NO: 15 - protein, 119 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSS

SEQ ID NO: 16 - protein, 107 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIK

SEQ ID NO: 17 - protein, 455 aa, Homo sapiens
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 18 - protein, 214 aa, Homo sapiens
AIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 449 aa, Homo sapiens
QVQLVQSGAEVKKPGASVKVSCETSGYTFTSYGISWVRQAPGHGLEWMGWISAYNGYTNYAQKLQGRVTMTTDTSTSTAYMELRSLRSDDTAVYYCARDLRGTNYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 20 - protein, 214 aa, Homo sapiens
DIQMTQSPSSVSASVGDRVTITCRASQGISNWLAWFQHKPGKAPKLLIYAASSLLSGVPSRFSGSGSGTDFTLTISSLQPEDFATYYCQQANSFPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC